(19) United States
(12) Patent Application Publication
(10) Pub. No.: US 2007/0160656 A1
(43) Pub. Date: Jul. 12, 2007

(54) Lipid platinum complexes and methods of use thereof

(76) Inventors: Krzysztof J. Dziewiszek, Acton, MA (US); Alemseged Truneh, Sudbury, MA (US)

Correspondence Address: JONES DAY, 222 EAST 41ST ST, NEW YORK, NY, 10017, US

(21) Appl. No.: 10/555,495

(22) PCT Filed: May 3, 2004
(86) PCT No.: PCT/US04/13727

§ 371 Date: Nov. 13, 2006

Related U.S. Application Data
(60) Provisional application No. 60/467,567, filed on May 2, 2003.

(51) Int. Cl. A61K 31/282; A61K 9/127; C07F 15/00

(52) U.S. Cl. 424/450; 554/71

(57) ABSTRACT

This invention provides novel lipid platinum complexes, liposomally encapsulated lipid platinum complexes, pharmaceutical compositions comprising a lipid platinum complex, and methods for treating cancer using a lipid platinum complex. Kits comprising a unit dosage form of a compound or composition of the invention are also provided.

This application claims the benefit of U.S. Provisional Application No. 60/467,567 filed May 2, 2003, which is incorporated herein by reference in its entirety.

### 1. FIELD OF THE INVENTION

The present invention relates to lipid platinum complexes, methods for making lipid platinum complexes, liposomally encapsulated lipid platinum complexes, compositions comprising a lipid platinum complex, and methods for treating cancer using a lipid platinum complex.

### 2. BACKGROUND OF THE INVENTION

Platinum coordination complexes were first identified as cytotoxic agents in 1965. cis-diamminedichloroplatinum (II) (cisplatin) is a clinically significant anticancer agent useful for the treatment of a broad spectrum of neoplastic diseases in humans. Loehrer et al., Ann. Int. Med. 100:704-713 (1984) However, long-term administration of cisplatin is limited by severe systemic toxicity, including emesis, nephrotoxicity, ototoxicity and neurotoxicity. Zwelling et al., “Platinum Complexes” in Pharmacologic Principles of Cancer Treatment, Ed. B. A. Chabner, Saunders, Philadelphia, Pa. (1982). In an attempt to modify the therapeutic index of cisplatin, numerous platinum analogs have been prepared and tested, typically with modest results. See M. C. Christian, “The Current Status of Platinum Analogs”, Seminars in Oncology, 1992, 19(6), 720-733.

cis-diammine(1,1-cyclobutanedicarboxylato)platinum (II) (carboplatin), is a second-generation platinum analog and is the only platinum drug other than cisplatin to enjoy widespread use in the clinic. Carboplatin is effective when used in place of cisplatin in established chemotherapeutic drug regimens and although less emetic, nephrotoxic, neurotoxic, and ototoxic than cisplatin, carboplatin has undesirable myelosuppressive properties that cisplatin does not. Go et al., J. Clin. Oncol. 1999, 17(1): 409-22. Oxaliplatin is a recently developed third-generation cisplatin analog with an 1,2-diaminocyclohexane (DACH) carrier ligand which has displayed clinical activity in a variety of tumor types and is not cross-resistant with cisplatin and carboplatin. Oxaliplatin acts synergistically with 5-fluorouracil (5-FU) in both 5-fluorouracil resistant and chemotherapy-naive disease and is currently being evaluated as a single-agent and in combination regiments against breast, lung and prostate cancer and non-Hodgkin's lymphoma. Misset et al., Crit Rev. Oncol. Hematol. 2000, 35(2): 75-93.

Other platinum analogs that have shown recent promise in clinical trials include NDDP (cis-bis-neodecanoato-trans-R,R-1,2-dicyclohexane platinum (II), U.S. Pat. No. 5,178,876; nedaplatin, Latorre et al., Int. J. Oncol. 2002, 21(1):179-86; JM335 (trans-amine-(cyclohexylaminedichlorodihydroxo)platinum(IV)), Kelland et al. J. Inorg. Biochem. 1999, 77(1-2):115-115; iproplatin, Martin, Clin. Breast Cancer 2001, 2(3):190-208; the dinuclear platinum complexes BBR3005 (trans-PtCl(NH₃)₂2H₂N(CH₂)₆NH₂)²⁺ and BBR3171 (cis-PtCl(NH₃)₂2H₂N(CH₂)₆NH₂)²⁺, and the trinuclear platinum complex BBR3464 (trans-PtCl(NH₃)2-2mu-trans-Pt(NH₃)2(H₂N(CH₂)₆NH₂)₂)⁴⁺, Roberts et al. J. Inorg. Biochem. 1999, 77(1-2):47-50; and the sterically hindered platinum complex, AMD473 (cis-aminedichloro(2-methylpyridine) platinum (II)), Holford, et al. J. Cancer 1998, 77(3):366-73.

U.S. Pat. No. 4,256,652 describes platinum complexes comprising resolved stereoisomers of 1,2-diaminocyclohexane (DACH), including cis-DACH, trans-R,R-DACH and trans-S,S-DACH. The trans-DACH ligated platinum complexes were typically more efficacious as anti-tumor agents than the analogous cis-DACH complexes.

Liposomes are lipid vesicles, which may form spontaneously upon addition of an aqueous solution to a dry lipid film, and can be used as drug carriers for both hydrophobic and hydrophilic drugs, said drugs being entrapped in the hydrophobic or hydrophilic compartments of the liposome, respectively. Mayhew et al., Liposomes, Ed., Marc J. Ostro, Marcel Dekker, Inc., New York, N.Y. (1983). Multilamellar liposomes are a class of multilayer lipid vesicles (MLVs) that are particularly suited for carrying hydrophobic drugs. When administered intravenously to animals and humans, MLVs concentrate in the liver, spleen and other organs rich in reticuloendothelial (RES) cells. Kasi et al. Int. J. Nucl. Med. Biol. 11:35-37 (1984); Lopez-Berestein et al., Cancer Drug Deliv., 1:199-205 (1984); and Lopez-Berestein et al., Cancer Res. 44:375-378 (1984).

Liposomes have been utilized for the in vitro delivery of anticancer agents (Mayhew et al., Liposomes, Ed. Ostro, Marcel Decker, Inc., New York, N.Y. (1983), immunomodulators and anti-fungal agents (Mehta et al., Immunology 51:517-527 (1984)). In vivo drug delivery using liposomes has been reported in both animals and humans. Lopez-Berestein et al., Clin Exp Metastasis 2:127-137(1984); Lopez-Berestein et al., J. Inf. Dis. 147:937-945 (1983); and Lopez-Berestein et al., J. Inf. Dis. 151:704-710 (1985).

Studies have reported that liposomal encapsulation of antineoplastic complexes can reduce drug-related toxicities, including doxorubicin-induced cardiotoxicity and cisplatin-induced nephrotoxicity. Forssen et al., Proc. Natl. Acad. Sci. 78:873-1877(1981); Olson et al., Bur. J. Cancer Clin. Oncol. 18:167-176 (1982); Gabizon et al., Cancer Res. 42:4734-4739 (1982); Herman et al., Cancer Res. 43:5427-5432 (1983); and Freise et al., Arch. Int. Pharmacodynamie Therapie 258:180-192 (1982). In addition, evidence suggests that liposomal incorporation of antineoplastic agents may increase the antitumor activity of these agents, possibly due to (a) extended release of the active agent (Mayhew et al., Ann. N.Y. Acad. Sci. 308:371-386, Patel, et al. Int. J. Cancer 34:717-723. (1984)); (b) increased uptake of the active agent by tumor cells; or (c) more selective organ distribution of the active agent. Gabizon et al., Cancer Res. 43:4730-4735 (1983); Mayhew et al., Cancer Drug Deliv. 1:43-58 (1983); and U.S. Pat. No. 4,330,534.

Liposomal cisplatin formulations have been prepared, but have been plagued by very poor encapsulation efficiency and by the relatively low stability of the resulting liposomal formulations. Freise et al., Arch. Int. Pharmacodynamie Therapie 258:180-192 (1982). The platinum complex L-NDDP is a liposomal formulation of the complex bis-neodecanoato-cis-1,2-diaminocyclohexane platinum (II) and is currently showing promise in clinical trials for pancreatic cancer, metastatic colorectal cancer and malignant mesothelioma. It is believed that L-NDDP is actually a prodrug which is activated intraliposomally to provide active platinum species which are responsible for the antitumor activity. Perez-Soler et al., Cancer Chemother. Pharmacol. (1994), 33:378-384.

Related art includes U.S. Pat. No. 5,041,581, “Hydrophobic Cis-Platinum Complexes Efficiently Incorporated into Liposomes; U.S. Pat. No. 5,117,022, “Hydrophobic Cis-Platinum Complexes Efficiently Incorporated into Liposomes; U.S. Pat. No. 5,178,876, “Hydrophobic Cis-Platinum Complexes Efficiently Incorporated into Liposomes;” U.S. Pat. No. 5,186,940, “Hydrophobic Cis-Platinum Complexes Efficiently Incorporated into Liposomes;” U.S. Pat. No. 5,384,127, “Stable Liposomal Formulation of Lipophilic Platinum complexes;” and U.S. Pat. No. 5,843,475, “Delivery and Activation Through Liposome Incorporation of Diaminocyclohexane Platinum (II) Complexes.”

European Patent Application No. 83306726.7 discloses platinum complexes having racemic DACH ligands and phosphatidyl ligands having fatty acid substituents. These complexes are described as largely insoluble in plasma and are preferably employed with lipid vesicle carriers. International Publication No. WO 98/33481 discloses a process for preparing various DACH-platinum complexes in the presence of phospholipids, but does not report the formation or isolation of platinum complexes with lipid ligation.

A barrier to the successful commercialization of liposomes has been the absence of adequate control over the large-scale manufacture of liposomes. Liposomes of diameter greater than 1 μM are less than ideal for certain therapeutic applications and therefore the reproducible production of submicron liposomes is desirable. U.S. Pat. No. 5,902,604 discloses that a preliposomal lyophilate comprising phospholipids and a surfactant can provide submicron liposomes upon reconstitution in aqueous media.

Despite the availability to the clinician of a variety of anticancer agents, traditional chemotherapy has many drawbacks (see, for example, Stockdale, 1998, “Principles Of Cancer Patient Management” in Scientific American Medicine, vol. 3, Rubenstein and Federman, eds., ch. 12, sect. 10). Almost all anticancer agents are toxic, and chemotherapy can cause significant, and often dangerous, side effects, including severe nausea, bone marrow depression, immunosuppression, etc. Additionally, many tumor cells are resistant or develop resistance to anticancer agents through multi-drug resistance. Therefore, there is a significant need in the art for novel agents with improved therapeutic indices that are useful for treating cancer and related proliferative diseases.

The recitation of any reference in Section 2 of this application is not an admission that the reference is prior art to the present invention.

### 3. SUMMARY OF THE INVENTION

The present invention relates to lipid platinum complexes and the use of lipid platinum complexes in the treatment of cancer.

Accordingly, in one aspect, the present invention provides a purified complex of formula (I) (a “lipid platinum complex”) as set forth below:

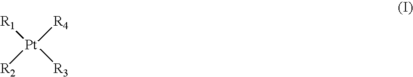

or pharmaceutically acceptable salts thereof,

wherein

R₁ and R₂ are independently —N(R₆)₂, —NH₃⁺; or R₁ and R₂ are each —NH₂ and join through a C₂-C₆ alkylene or C₃-C₇ cycloalkylene group to form a bidentate diamine ligand, optionally substituted with one or more R₇;

R₃ is a lipid ligand, with the proviso that R₃ cannot be a phosphatidic acid;

R₄ is a lipid ligand, an inorganic ligand, —CN or —OC(O)R₅, with the proviso that R₄ cannot be a phosphatidic acid;

R₅ is C₁-C₂₄ alkyl;

each R₆ is independently —H, —C₁-C₆ alky, —C₃-C₇ cycloalkyl or -aryl; and

each R₇ is independently —C₁-C₆ alkyl, —C₃-C₇ cycloalkyl or -aryl.

The invention also provides liposomes comprising a lipid platinum complex (a “liposomal lipid platinum complex”).

Also provided by the invention is a method for treating cancer comprising administering to a subject in need of such treatment an amount of a lipid platinum complex or a liposomal lipid platinum complex effective to treat cancer.

The invention also includes pharmaceutical compositions that comprise an amount of a lipid platinum complex or a liposomal lipid platinum complex effective to treat cancer, and a pharmaceutically acceptable carrier or vehicle. The compositions are useful for treating cancer. The invention includes a lipid platinum complex when provided as a pharmaceutically acceptable salt.

In another embodiment, the invention includes methods for making lipid platinum complexes.

The present invention also provides kits comprising a container which contains a lipid platinum complex or a liposomal lipid platinum complex.

The details of the invention are set forth in the accompanying description and examples below. Although any methods and materials similar or equivalent to those described herein can be used in the practice or testing of the present invention, illustrative methods and materials are now described. Other features, objects, and advantages of the invention will be apparent from the description and from the claims. In the specification and the appended claims, the singular forms also include the plural unless the context clearly dictates otherwise.

### 4. DETAILED DESCRIPTION OF THE INVENTION

This invention provides a novel class of lipid platinum complexes according to Formula I, as set forth below: 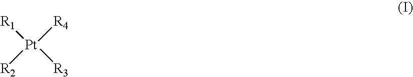
and pharmaceutically acceptable salts, solvates and hydrates thereof,

wherein

R₁ and R₂ are independently —N(R₆)₂, —NH₃⁺; or R₁ and R₂ are each —NH₂ and join through a C₂-C₆ alkylene or C₃-C₇ cycloalkylene group to form a bidentate diamine ligand, optionally substituted with one or more R₇;

R₃ is a lipid ligand, with the proviso that R₃ cannot be a phosphatidic acid;

R₄ is a lipid ligand, an inorganic ligand, —CN or OC(O)R₅, with the proviso that R₄ cannot be a phosphatidic acid;

R₅ is C₁-C₂₄ alkyl;

each R₆ is independently —H, —C₁-C₆ alkyl, —C₃-C₇ cycloalkyl or -aryl; and

each R₇ is independently —C₁-C₆ alkyl, —C₃-C₇ cycloalkyl or -aryl.

In one embodiment the lipid platinum complexes are in purified form.

### 4.1 DEFINITIONS

The term “C₁-C₆ alkyl” as used herein refers to a straight- or branch-chain, saturated or unsaturated hydrocarbon having from 1 to 6 carbon atoms. Representative C₁-C₆ alkyl groups include, but are not limited to methyl, ethyl, propyl, isopropyl, butyl, sec-butyl, tert-butyl, pentyl, isopentyl, neopentyl, hexyl, isohexyl, neohexyl, ethylenyl, propylenyl, 1-butenyl, 2-butenyl, 1-pentenyl, 2-pentenyl, 1-hexenyl, 2-hexenyl, 3-hexenyl, acetylenyl, pentynyl, 1-butynyl, 2-butynyl, 1-pentynyl, 2-pentynyl, 1-hexynyl, 2-hexynyl and 3-hexynyl.

The term “alkoxy” as used herein refers to a —O—(C₁-C₆ alkyl) group. Representative alkoxy groups include, but are not limited to methoxy, ethoxy, propoxy, isopropoxy, butoxy, sec-butoxy, tert-butoxy, pentyloxy, isopentyloxy, neopentyloxy, hexyloxy, isohexyloxy and neohexyloxy.

The term “C₁-C₂₄ alkyl” as used herein refers to a straight- or branch-chain, saturated or unsaturated hydrocarbon having from 1 to 24 carbon atoms. Representative C₁-C₂₄ alkyl groups include, but are not limited to, methyl, ethyl, propyl, butyl, pentyl, hexyl, heptyl, octyl, nonyl, decyl, undecyl, dodecyl, isopropyl, isobutyl, sec-butyl and tert-butyl, isopentyl, neopentyl, isohexyl, neohexyl, isoheptyl, neoheptyl, isooctyl, neooctyl, isononyl, neononyl, isodecyl, neodecyl, myristoyl, oleoyl, palmitoyl, stearoyl, lauroyl and caproyl. A C₁-C₂₄ alkyl group can be unsubstituted or optionally substituted with one or more —C₁-C₆-alkyl, —C₃-C₇ cycloalkyl, -alkoxy, -aryl, -heterocyclic, -halo, —CN, —COOH, —COOR₆, —OC(O)R₆, —NH₂, —C(O)R₆, —CHO, NHR₆, —N(R₆)₂, NHC(O)R₆ or —C(O)NHR₆ groups wherein R₆ is —H, —C₁-C₆ alkyl, —C₃-C₇ cycloalkyl or -aryl.

As used herein, the term “alkylcarboxylato” refers to a group having the structure: 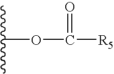
wherein R₅ is a C₁-C₂₄ alkyl group.

“C₂-C₆ alkylene” refers to a divalent, straight- or branch-chain, saturated hydrocarbon having from 2 to 6 carbon atoms.

The term “aryl” as used herein refers to a phenyl group or a naphthyl group.

The term “bidentate diamine ligand” as used herein refers to ligands of the general formula:
NH₂—X—NH₂
wherein X is a C₂-C₆ alkylene or C₃-C₇ cycloalkylene group which links the two NH₂ groups. Such a bidentate ligand coordinates to the platinum via the two NH₂ groups, each of which occupies a separate coordination site on the metal. A bidentate diamine ligand can be chiral or achiral. Representative bidentate diamine ligands include, but are not limited to, trans-R,R-1,2-diaminocyclohexane, trans-S,S-1,2-diaminocyclohexane, cis-1,2-diaminocyclohexane and 1,2-ethylenediamine, (1R,2R)—(+)-1,2-diphenylethylenediamine and (1S,2S)—(−)-1,2-diphenylethylenediamine

The term “C₃-C₇ cycloalkyl” as used herein is a 3-, 4-, 5-, 6- or 7-membered saturated or unsaturated non-aromatic carbocyclic ring. Representative cycloalkyls include, but are not limited to, cyclopropyl, cyclobutyl, cyclopentyl, cyclopentadienyl, cyclohexyl, cyclohexenyl, cycloheptyl, cycloheptanyl, 1,3-cyclohexadienyl, 1,4-cyclohexadienyl, 1,3-cycloheptadienyl, and 1,3,5-cycloheptatrienyl.

“C₃-C₇ cycloalkylene” refers to a 3-, 4-, 5-, 6- or 7-membered divalent, saturated or unsaturated non-aromatic carbocyclic ring.

The term “halo” as used herein refers to —F, —Cl, —Br or —I.

The term “inorganic ligand” as used herein refers to a ligand that does not comprise a carbon-containing organic functional group. Representative examples of inorganic ligands include, but are not limited to, Cl⁻, Br⁻, I⁻, F⁻, NO₃⁻, OH⁻, H₂O, HCO₃⁻ and HSO₄⁻.

The term “lipid platinum complex” as used herein, refers to a tetracoordinate platinum complex of formula (I) as described herein. In a preferred embodiment, a lipid platinum complex is in purified form.

As used herein, the term “purified” means that when isolated (e.g., from other components of a synthetic organic chemical reaction mixture), the isolate contains at least 50%, at least 55%, at least 60%, at least 65%, at least 70%, at least 75%, at least 80%, at least 85%, at least 90%, at least 95% or at least 98% of a lipid platinum complex of the invention by weight of the isolate. In a preferred embodiment, the isolate contains at least 95% of a lipid platinum complex of the invention by weight of the isolate.

The following abbreviations are used herein and have the indicated definitions: DACH is 1,2-diaminocyclohexane; DMPC is dimyristoyl phosphatidyl choline; DMPG is dimyristoyl phosphatidyl glycerol; DMSO is dimethylsulfoxide; EtOH is ethyl alcohol; HPLC is high pressure liquid chromatography; L-NDDP is NDDP which has been encapsulated in a liposome; NDDP is cis-bis-neodecanoato-trans-R,R-1,2-dicyclohexane platinum (II); and MLV is multilamellar lipid vesicle.

### 4.2 LIPID PLATINUM COMPLEXES OF THE INVENTION

The lipid platinum complexes of the invention are tetracoordinate platinum (II) complexes in which two adjacent coordination sites (represented by R₁ and R₂ in Formula I) are independently occupied by an amine or an amine (NH₃⁺) ligand, or alternately, R₁ and R₂ together represent a single bidentate diamine ligand. The third coordination site (R₃ of Formula I) is occupied by a lipid ligand, while the final coordination site (R₄ of Formula I) can be occupied by a lipid ligand, an inorganic ligand or an organic ligand, with the proviso that phosphatidic acids are excluded from the present invention as lipid ligands.

The present invention further encompasses pharmaceutically acceptable salts of the lipid platinum complexes of the invention, including both organic and inorganic salts of the lipid platinum complexes of the invention. The lipid platinum complexes of the invention contain at least one amino group, and accordingly, it is possible to form acid addition salts with an amino group of a lipid platinum complex of the invention. Preferred salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion can be any organic or inorganic moiety that stabilizes a charge which may be present on a lipid platinum complex of the invention. Furthermore, a pharmaceutically acceptable salt may have more than one charged atom in its structure. In instances where multiple charged atoms are part of the pharmaceutically acceptable salt, said salt can have multiple counterions. Hence, a pharmaceutically acceptable salt of a lipid platinum complex can have one or more charged atoms and/or one or more counterions.

### 4.2.1 Amine/Ammine Ligands

R₁ and R₂ are independently an amine or ammine (NH₃⁺) ligand. The amine ligands of the invention are represented by the formula —N(R₆)₂ where each R₆ is independently —H, —C₁-C₆ alkyl, —C₃-C₇ cycloalkyl or -aryl. In an alternate embodiment, R₁ and R₂ are each —NH₂ and join through a C₂-C₆ alkylene or C₃-C₇ cycloalkylene group to form a bidentate diamine ligand, optionally substituted with R₇. Bidentate diamine ligands useful in the invention include, but are not limited to, trans-R,R-1,2-diaminocyclohexane, trans-S,S-1,2-diaminocyclohexane, cis-1,2-diaminocyclohexane and 1,2-ethylenediamine.

In one embodiment, a lipid platinum complex comprises a bidentate diamine ligand.

In a specific embodiment, a lipid platinum complex comprises a bidentate 1,2-ethylenediamine ligand. In another embodiment the bidentate 1,2-ethylenediamine ligand is 1,2-diphenylethylenediamine. In another embodiment, the 1,2-ethylenediamine ligand is (1R, 2R)—(+)-1,2-diphenylethylenediamine or (1S,2S)—(−)-1,2-diphenylethylenediamine.

In a particular embodiment, a lipid platinum complex comprises a bidentate 1,2-diaminocyclohexane ligand.

In a preferred embodiment, a lipid platinum complex comprises a bidentate trans-R,R-1,2-diaminocyclohexane ligand.

### 4.2.2 Lipid Ligands

Lipids are useful herein as addition moieties to platinum and the lipid platinum complexes of the invention comprise a tetracoordinate platinum complex having one or two lipid ligands. For convenience, and to differentiate between (i) lipids that can be used as ligands on a lipid platinum complex; and (ii) lipids that can be used as liposomal components of a liposomal lipid platinum complex, such lipids will be referred to herein as “lipid ligands” and “liposomal lipid components,” respectively.

Lipid ligands useful in the present invention include, but are not limited to, phospholipids, glycolipids, glycosphingolipids and sterols. Representative examples of glycolipids useful as lipid ligands include, but are not limited to, glycosphingolipids, such as ceramides, cerebrosides and gangliosides. Representative examples of sterols useful as lipid ligands include, but are not limited to, cholesterol. Phosphatidic acids are excluded from use as lipid ligands in the present invention.

In one embodiment, a lipid platinum complex has one lipid ligand.

In another embodiment, a lipid platinum complex has two lipid ligands, which may be the same or different.

In one embodiment, the lipid ligand is a glycolipid.

In a specific embodiment, the lipid ligand is a cerebroside, ganglioside or cardiolipin.

In another embodiment, the lipid ligand is a sterol.

In a specific embodiment, the lipid ligand is cholesterol.

In a preferred embodiment, the lipid ligand is a phospholipid.

In a specific embodiment, a lipid platinum complex has one lipid ligand which is a phospholipid.

In another specific embodiment, a lipid platinum complex has two lipid ligands which are phospholipids.

Preferred phospholipids useful in the invention as lipid ligands have the structure:

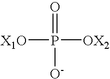

wherein

X₁ is 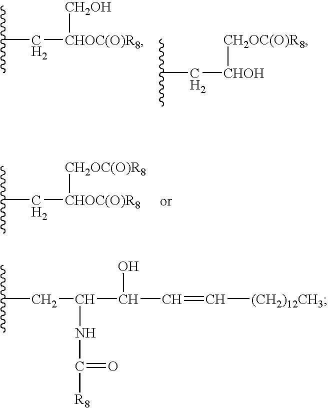
X₂ is -C₁-C₆ alkylene)-(NR₉)₂, —(C₁-C₆ alkylene)-OH, (C₁-C₆ alkylene)-N⁺(R₉)₃, —C₁-C₆ alkylene)-CH(OH)CH₂OH, —CH₂CH[—N(R₉)₃]COOH or -inositol-1-yl;

each occurrence of R₈ is independently —H or C₁-C₆ alkyl;

each occurrence of R₉ is independently —H or —C₁-C₂₄ alkyl.

Other phospholipids useful in the invention as lipid ligands include, but are not limited to, phosphatidyl cholines, phosphatidyl glycerols, phosphatidyl ethanolamines and sphingolipids, particularly sphingomyelin.

In one embodiment, phospholipids useful in the invention as lipid ligands include, but are not limited to, dimyristoyl phosphatidyl choline (DMPC), egg phosphatidyl choline, dilauryloyl phosphatidyl choline, dipalmitoyl phosphatidyl choline, distearoyl phosphatidyl choline, 1-myristoyl-2-palmitoyl phosphatidyl choline, 1-palmitoyl-2-myristoyl phosphatidyl choline, 1-palmitoyl-2-stearoyl phosphatidyl choline, 1-stearoyl-2-palmitoyl phosphatidyl choline, dioleoyl phosphatidyl choline, dimyristoyl phosphatidyl glycerol (DMPG), dilauryloyl phosphatidyl glycerol, dioleyl phosphatidyl glycerol, dipalmitoyl phosphatidyl glycerol, distearoyl phosphatidyl glycerol, 1-myristoyl-2-palmitoyl phosphatidyl glycerol, 1-palmitoyl-2-myristoyl phosphatidyl glycerol, 1-palmitoyl-2-stearoyl phosphatidyl glycerol, 1-stearoyl-2-palmitoyl phosphatidyl glycerol, dioleoyl phosphatidyl glycerol, dimyristoyl phosphatidyl ethanolamine, dipalmitoyl phosphatidyl ethanolamine, brain sphingomyelin, dipalmitoyl sphingomyelin, and distearoyl sphingomyelin.

In one embodiment, the lipid ligand is a phosphatidyl glycerol.

In another embodiment, the lipid ligand is a phosphatidyl choline.

In a preferred embodiment, the lipid ligand is dimyristoyl phosphatidyl glycerol or dimyrisoyl phosphatidyl choline.

Phospholipids that can be used according to the invention as lipid ligands also include, but are not limited to, synthetic phospholipids which can be unsubstituted or which may have one or more aliphatic moieties which can be independently substituted with one or more of —OH, —C₁-C₆ alkyl, —CN, —NO₂, —C₃-C₇ cycloalkyl, -aryl, -halo, -alkoxy, —NH₂, —NH—(C₁-C₆ alkyl), —N—(C₁-C₆ alkyl)₂, —C(O)OH, —C(O)O—(C₁-C₆ alkyl), —O—C(O)—(C₁-C₆ alkyl), —C(O)—(C₁-C₆ alkyl), —C(O)NH₂, —C(O)NH—(C₁-C₆ alkyl) or —C(O)N—(C₁-C₆ alkyl)₂.

Phospholipid salts can also serve as platinum ligands in the present invention. Examples of phospholipid salts useful as platinum ligands in the invention include, but are not limited to ammonium salts; alkali metal salts, such as potassium, sodium, lithium, calcium or magnesium salts; and other metal salts, such as silver or mercury salts.

### 4.2.3 Other Ligands

The lipid platinum complexes can also comprise non-amino and/or non-lipid ligands.

Other ligands useful in the invention include, but are not limited to, inorganic ligands, including, but not limited to, Cl⁻, Br⁻, I⁻, F⁻, NO₃⁻, OH⁻, H₂O, HCO₃⁻ and HSO₄⁻; and organic ligands, including but not limited to, —CN and alkylcarboxylato groups of the formula —OC(O)R₅, wherein R₅ is C₁-C₂₄ alkyl. Representative examples of alkylcarboxylato groups useful as platinum ligands in the invention include, but are not limited to, neopentanoato, neoheptanoato, neooctanoato, neononanoato, neodecanoato, cyclopentenecarboxylato, 2,2-dimethyloctanoato, 2,2-diethyl-4-methylpentanoato, 2-ethylhexanoato, 2-ethylbutyrato, 2-propylpentanoato, 2-methyl-2-ethylheptanoato, 2,2-diethylhexanoato, 2,2-dimethyl-4-ethylhexanoato, laurato, myristato and 2,2,4,4-tetramethylpentanoato.

In one embodiment, the ligand is an alkylcarboxylato group having from 6 to 14 carbon atoms.

In another embodiment, the ligand is an alkylcarboxylato group having from 9 to 11 carbon atoms.

In a specific embodiment, the ligand is an alkylcarboxylato group having 10 carbon atoms.

In another embodiment, a lipid platinum complex of the invention has a ligand which is an alkylcarboxylato group of the formula —OC(O)R₅, wherein R₅ is a branched C₁-C₂₄ alkyl group.

In still another embodiment, a lipid platinum complex has a ligand which is an alkylcarboxylato group of the formula —OC(O)R₅, wherein R₅ is a linear C₁-C₂₄ alkyl group.

In a preferred embodiment, a lipid platinum complex of the invention has a ligand which is a neodecanoato group.

It will be appreciated by one skilled in the art of organic chemistry that certain organic ligand groups disclosed herein can have one or more chiral centers. The invention is assumed to encompass all possible stereoisomers of any ligands which possess one or more chiral centers.

### 4.3 Preparation of Lipid Platinum Complexes

Lipid platinum complexes of formula (I) can be prepared via the synthetic procedure outlined below in Scheme 1. It will be apparent to one skilled in the art how to prepare the scope of the lipid platinum complexes of the invention by choice of proper and relevant starting materials, synthetic intermediates and reagents.

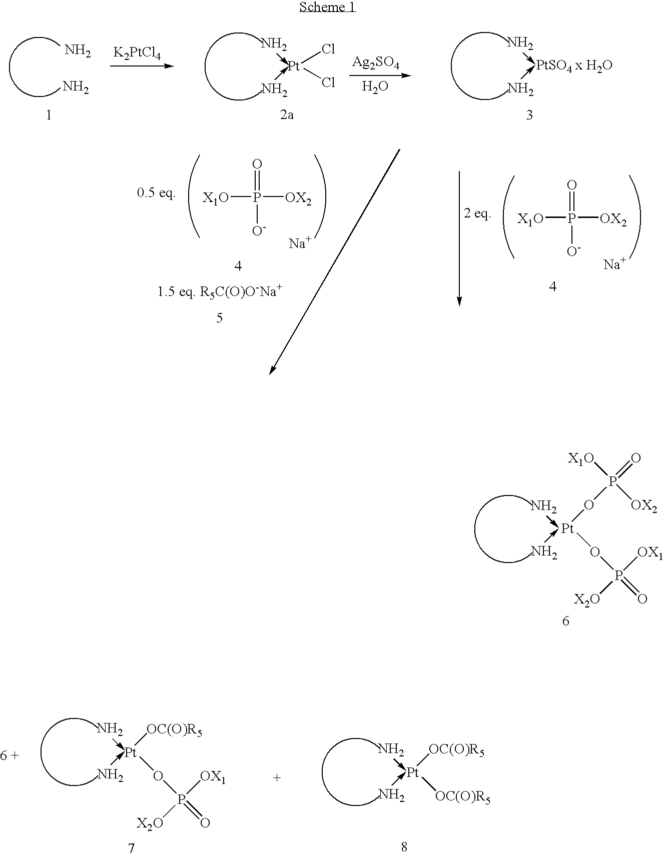

In a typical procedure, an amine, represented here by the generic bidentate diamine 1, is treated with potassium tetrachloroplatinate to yield a diaminodichloroplatinum complex of formula 2a, which is then treated with silver sulfate and water to provide the intermediate diamino sulfatoplatinum (II) monohydrate of formula 3. Intermediate 3 can then be reacted with a stoichiometric excess of a reactive lipid, such as a phospholipid of formula 4, to provide the diamino-bis-phospholipid platinum complex of formula 6. In a specific embodiment, instead of reacting intermediate 3 with a stoichiometric excess of a single lipid, intermediate 3 may be reacted simultaneously with one equivalent each of two different lipids in order to provide a lipid platinum complex having two different lipid ligands. The complex of formula 3 can alternately be reacted with a sub-stoichiometric amount of a lipid, such as 4, in the presence of an excess of another reactive ligand, such as the alkali metal salt of an inorganic ligand or the alkali metal salt of an alkylcarboxylate 5, to yield the platinum complex of formula 7, which contains both lipid and non-lipid ligation.

In an alternate embodiment, lipid platinum complexes of formula (I) can be prepared via the synthetic procedure outlined below in Scheme 2.

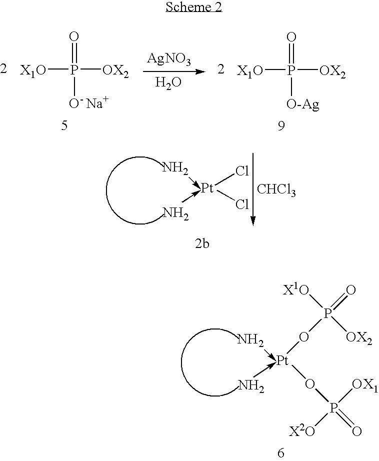

In Scheme 2, two equivalents of the phospholipid of formula 5 are reacted with two equivalents of AgNO₃ to provide two equivalents of the phospholipid of formula 9. Two equivalents phospholipid of formula 9 are then reacted with one equivalent of platinum complex 2b in chloroform to yield the diamino-bis-phospholipid platinum complex of formula 6.

In a specific embodiment of Scheme 2, the generic bidentate platinum complex 2b is cis-[trans-(1R,2R)-1,2-diaminocyclohexane]diiodoplatinum(II) (10), the generic sodium phospholipid 5 is the sodium salt of 1,2-dimyristoyl-sn-glycero-3-phospho(rac-1-glycerol) (13), and the complex formed of complex 10 and silver complex 9 is cis-bis[1,2-dimyristoyl-sn-glycero-3-phospho(rac-1-glycerol)][trans-(1R,2R)-1,2-diaminocyclohexane]platinum(II), (14).

General Procedure for the Preparation of a Diaminodichloro Platinum Complex of Formula 2:

An approximately 0.5M solution of a diamine I in water is added slowly to a filtered solution of K₂PtCl₄ (1.05 eq.) in water at room temperature. The resulting mixture is allowed to stand at room temperature for about 16 hours, after which time a colored precipitate may appear. The precipitate is filtered and washed with water until the filtrate is no longer reactive toward AgNO₃, and the filtrate is then washed sequentially with ethanol and acetone. The precipitate is allowed to dry in the filtration funnel for about 1 hour and is then dried in vacuo to provide a complex of formula 2a.

General Procedure for the Preparation of a Diamino Sulfatoplatinum Complex of Formula 3:

An approximately 0.03M solution of Ag₂SO₄ (about 0.97 eq.) in water is taken up in a reaction vessel that is protected from light and the resulting aqueous solution is warmed to about 40° C. The complex of formula 2a is added, and the reaction is allowed to stir for about 48 hours at room temperature. The reaction mixture is then filtered through a ˜1 cm pad of Celite and the Celite is washed with water. The filtrate is then concentrated in vacuo and the resulting solid residue is dried in vacuo to provide a complex of formula 3.

General Procedure for the Preparation of a Diamino-Bis-Phospholipid Platinum Complex of Formula 6:

To an approximately 0.1M stirred solution of the complex of formula 3 in water is added an approximately 0.5 M solution of a phospholipid sodium salt 4 (about 2.0 eq.) in a mixture of H₂O:EtOH (about 8:1 by volume). The reaction mixture is stirred for about 24 hours and the resulting precipitate is filtered, washed with water and dried in vacuo to provide the complex of formula 6, which can be further purified using column chromatography or HPLC.

General Procedure for the Preparation of a Diamino-Phospholipid Platinum Complex of Formula 7:

To an approximately 0.5M stirred solution of complex of formula 3 in water is added a solution of a phospholipid sodium salt 4 (about 0.5 eq.) and an alkoxycarbonyl sodium salt 5 (about 1.5 eq.) in a mixture of H₂O:EtOH (about 8:1 by volume). The reaction mixture is stirred for about 24 hours and the resulting precipitate is filtered, washed with water and dried in vacuo to provide the complex of formula 7, which can be further purified using column chromatography or HPLC.

Complexes of the invention that can be prepared using the method of Scheme 1 include, but are not limited to, cis-bis[1,2-dimyristoyl-sn-glycero-3-phospho(rac-1-glycerol)trans-(1R,2R)-1,2-diaminocyclohexane]platinum (II); cis-bis[1,2-dimyristoyl-sn-glycero-3-phospho(rac-1-glycerol)trans-(1S,2S)-1,2-diaminocyclohexane]platinum (II); cis-bis[1,2-dimyristoyl-sn-glycero-3-phospho(rac-1-glycerol)cis-1,2-diaminocyclohexane]platinum (II); cis-[1,2-dimyristoyl-sn-glycero-3-phospho(rac-1-glycerol)(neo-decanoato)trans-(1R,2R)-1,2-diaminocyclohexane]platinum (II); cis-[1,2-dimyristoyl-sn-glycero-3-phospho(rac-1-glycerol)(neo-decanoato)trans-(1S,2S)-1,2-diaminocyclohexane]platinum (II); and cis-[1,2-dimyristoyl-sn-glycero-3-phospho(rac-1-glycerol)(neo-decanoato)cis-1,2-diaminocyclohexane]platinum (II).

In one embodiment, the lipid platinum complex is cis-bis[1,2-dimyristoyl-sn-glycero-3-phospho(rac-1-glycerol)]trans-(1R,2R)-1,2-diaminocyclohexane]platinum(II).

In another embodiment, the lipid platinum complex is cis-[1,2-dimyristoyl-sn-glycero-3-phospho(rac-1-glycerol)](neo-decanoato)trans-(1R,2R)-1,2-diaminocyclohexane]platinum(II).

In a preferred embodiment, the lipid platinum complexes are in purified form.

The present invention also relates to methods for making a compound of formula (I). In one embodiment, the invention relates to a method making a compound of formula (I), comprising allowing a complex of formula (II), 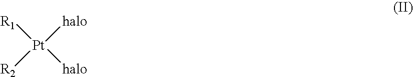
to react with at least about 2 molar equivalents of a compound of formula (III), 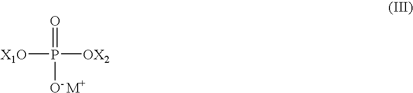
where

halo is —F, —Cl, —Br, —I or —At;

M⁺ is Li⁺, Na⁺, K⁺, Rb⁺, Cs⁺ or Ag⁺; and

R₁, R₂, R₃, R₄, X₁ and X₂ are as defined above.

In one embodiment, the invention relates to a method for making a compound of formula (I) where R₃ is a phospholipid.

In one embodiment, the invention relates to a method for making a compound of formula (I) where R₄ is a phospholipid.

In one embodiment, the invention relates to a method for making a compound of formula (I) where R₃ and R₄ are each independently a phospholipid

In one embodiment, the invention relates to a method for making a compound of formula (I) where R₁ and R₂ join to form a bidentate diamine ligand.

In an other embodiment, the invention relates to a method for making a compound of formula (I) where R₁ and R₂ join to form a bidentate diamine ligand and the bidentate diamine ligand is trans-R,R-1,2-diaminocyclohexane, trans-S,S-1,2-diaminocyclohexane, cis-1,2-diaminocyclohexane or 1,2-ethylenediamine.

In an other embodiment, the invention relates to a method for making a compound of formula (I) where R₁ and R₂ join to form trans-R,R-1,2-diaminocyclohexane, and R₃ and R₄ are each 1,2-dimyristoyl-sn-glycero-3-phospho(rac-1-glycerol).

In an other embodiment, the invention relates to a method for making a compound of formula (I) where R₁ and R₂ join to form trans-R,R-1,2-diaminocyclohexane, R₃ and R₄ are each 1,2-dimyristoyl-sn-glycero-3-phospho(rac-1-glycerol), and M⁺ is Ag⁺.

### 4.4 Liposomal Lipid Platinum Complexes

In a preferred embodiment, the invention provides liposomes comprising a lipid platinum complex and a liposomal lipid component, as well as the preparation and use of these liposomes. For convenience, these liposomal formulations are herein referred to as “liposomal lipid platinum complexes.” The liposomal lipid platinum complexes of the invention are useful for treating cancer.

In a preferred embodiment, the liposomal lipid platinum complexes of the invention comprise a lipid platinum complex which is in purified form.

In addition to a lipid platinum complex, the liposomal lipid platinum complexes of the invention also comprise a liposome containing the lipid platinum complex. The lipids which comprise the liposome of a liposomal lipid platinum complex are herein referred to as “liposomal lipid components.” Lipid useful in the present invention as liposomal lipid components include, but are not limited to, phospholipids, glycolipids, glycosphingolipids and sterols. Representative examples of glycolipids useful as liposomal lipid components include, but are not limited to, glycosphingolipids, such as ceramides, cerebrosides and gangliosides. Representative examples of sterols useful as liposomal lipid components include, but are not limited to, cholesterol.

In one embodiment, the liposomes of the present invention comprise two or more different liposomal lipid components.

In a specific embodiment, the liposomes of the present invention comprise two different liposomal lipid components.

In a preferred embodiment, the liposomal lipid component is a phospholipid. Phospholipids useful in the invention as liposomal lipid components include, but are not limited to, phosphatidyl cholines, phosphatidyl glycerols, phosphatidyl ethanolamines and sphingolipids, particularly sphingomyelin.

Representative examples of phospholipids useful as liposomal lipid components of the invention include, but are not limited to, dimyristoyl phosphatidyl choline (DMPC), egg phosphatidyl choline, dilauryloyl phosphatidyl choline, dipalmitoyl phosphatidyl choline, distearoyl phosphatidyl choline, 1-myristoyl-2-palmitoyl phosphatidyl choline, 1-palmitoyl-2-myristoyl phosphatidyl choline, 1-palmitoyl-2-stearoyl phosphatidyl choline, 1-stearoyl-2-palmitoyl phosphatidyl choline, dioleoyl phosphatidyl choline, dimyristoyl phosphatidyl glycerol (DMPG), dilauryloyl phosphatidyl glycerol, dioleyl phosphatidyl glycerol, dipalmitoyl phosphatidyl glycerol, distearoyl phosphatidyl glycerol, 1-myristoyl-2-palmitoyl phosphatidyl glycerol, 1-palmitoyl-2-myristoyl phosphatidyl glycerol, 1-palmitoyl-2-stearoyl phosphatidyl glycerol, 1-stearoyl-2-palmitoyl phosphatidyl glycerol, dioleoyl phosphatidyl glycerol, dimyristoyl phosphatidyl ethanolamine, dipalmitoyl phosphatidyl ethanolamine, brain sphingomyelin, dipalmitoyl sphingomyelin, and distearoyl sphingomyelin.

Preferred phospholipids which are useful as liposomal lipid components of the invention, include, but are not limited to, phosphatidylglycerols and phosphatidylcholines. The most preferred phosphatidylglycerol is one consisting essentially of DMPG and the most preferred phosphatidylcholine is one consisting essentially of DMPC. In a preferred embodiment, the liposomal lipid compositions of the present invention have liposomes comprising a mixture of DMPG and DMPG as liposomal lipid components, preferably in a molar ratio between 1 to 10 and 10 to 1, more preferably in a molar ratio of 3 to 7.

In a specific embodiment, the liposomal lipid platinum complexes of the present invention contain the lipid platinum complex and the liposomal lipid component in a molar ratio between 1 to 2 and 1 to 30. In one embodiment, the lipid platinum complex and the liposomal lipid component are present in a molar ratio between 1 to 2 and 1 to 7. In another embodiment, the lipid platinum complex and the liposomal lipid component are present in a molar ratio between 1 to 3 and 1 to 5. The foregoing molar ratios are particularly preferred when the liposomal lipid component is DMPC, DMPG or a combination thereof.

When the liposome of a liposomal lipid platinum complex does not comprise either DMPC or DMPG, the liposomal lipid platinum complexes of the present invention may contain the lipid platinum complex and the liposomal lipid component in a molar ratio between 1 to 2 and 1 to 30, preferably between 1 to 5 and 1 to 20, most preferably between 1 to 10 and 1 to 15.

The liposomes of the liposomal lipid platinum complexes can be multilamellar, unilamellar or have an undefined lamellar construction. A pharmaceutical composition comprising an amount of a liposomal lipid platinum complex effective to treat cancer, and a pharmaceutically acceptable carrier or vehicle can be administered for the treatment of cancer.

The liposomal lipid platinum complexes of the invention, in addition to comprising a lipid platinum complex and a liposomal lipid component, may further comprise an additional anticancer agent other than a compound of the invention (herein referred to as an “additional anticancer agent”) such that a lipid platinum complex of the invention and an additional anticancer agent are entrapped in the same liposome. Additional anticancer agents include, but are not limited to those listed herein below in section 4.6.3.

In one embodiment the additional anticancer agent entrapped in a liposomal lipid platinum complex of the invention is gemcitabine, capecitabine or 5-fluorouracil.

The preparation of liposomes comprising a tetracoordinate platinum(II) species and a phospholipid is described in U.S. Pat. No. 5,041,581, which is incorporated by reference herein in its entirety.

The liposomal lipid platinum complexes of the invention can further comprise a surfactant, said surfactant being nonionic, anionic, or cationic. Such liposomes can have median diameters of less than 1 μM. Examples of surfactants useful in the invention include, but are not limited to, sorbitan polyoxyethylene carboxylates, such as sorbitan polyoxyethylene monooleate and sorbitan polyoxyethylene monolaurate; sorbitan esters of common fatty acids, such as sorbitan monooleate, sorbitan monopalmitate and sorbitan monolaurate; polyoxyethylene ethers, such as polyoxyethylene monolauryl ether, polyoxyethylene monopalmityl ether, polyoxyethylene monostearyl ether and polyoxyethylene monooleyl ether; and block copolymers, such as those comprising ethylene oxide and propylene oxide.

Liposomal lipid platinum complexes of the invention having a submicron diameter can be prepared by adding a surfactant to a solution of the liposomal phospholipid component(s) and a lipid platinum complex. The surfactant can be present in an amount between 0.01 mole % to 5 mole % of the total amount of the liposomal lipid component(s). In one embodiment, the surfactant is present in an amount between 0.5 mole % and 4 mole % of the total amount of the liposomal lipid component(s). In a preferred embodiment, the surfactant is present in an amount between 1.5 mole % and 3 mole % of the total amount of the liposomal lipid component(s). The preparation of submicron diameter liposomes comprising an anticancer agent, a surfactant and a phospholipid is described in U.S. Pat. No. 5,902,604, which is incorporated by reference herein in its entirety.

In one embodiment, the surfactant is a nonionic surfactant.

In another embodiment, the nonionic surfactant is a polyoxyethylene sorbitan carboxylate.

In a specific embodiment, the nonionic surfactant is polyoxyethylene sorbitan monooleate.

In another specific embodiment, the nonionic surfactant is polyoxyethylene sorbitan monolaurate.

The submicron diameter liposomal lipid platinum complexes of the invention can possess valuable pharmacological properties. Submicron liposomal formulations do not occlude capillaries of the circulatory system of a subject and are therefore particularly useful in parenteral and, more particularly, intravenous modes of administration.

Thus, submicron diameter liposomal lipid platinum compounds of the present invention are especially useful in treating cancer.

### 4.5 Pharmaceutical Compositions and Therapeutic Administration

In other aspects, the present invention provides a pharmaceutical composition comprising an effective amount of a compound of the invention and a pharmaceutically acceptable carrier or vehicle. For ease of reference, the lipid platinum complexes of the invention and the liposomal lipid platinum complexes of the invention are each considered a “compound of the invention”. The pharmaceutical compositions are suitable for veterinary or human administration.

The pharmaceutical compositions of the present invention can be in any form that allows for the composition to be administered to a subject, said subject preferably being an animal, including, but not limited to a human, mammal, or non-human animal, such as a cow, horse, sheep, pig, fowl, cat, dog, mouse, rat, rabbit, guinea pig, etc., and is more preferably a mammal, and most preferably a human.

The compositions of the invention can be in the form of a solid, liquid or gas (aerosol). Typical routes of administration may include, without limitation, oral, topical, parenteral, sublingual, rectal, vaginal, ocular, and intranasal. Parenteral administration includes subcutaneous injections, intravenous, intramuscular, intraperitoneal, intrapleural, intrasternal injection or infusion techniques. Preferably, the compositions are administered parenterally, most preferably intravenously. Pharmaceutical compositions of the invention can be formulated so as to allow a compound of the invention to be bioavailable upon administration of the composition to a subject. Compositions can take the form of one or more dosage units, where for example, a tablet can be a single dosage unit, and a container of a compound of the invention in aerosol form can hold a plurality of dosage units.

Materials used in preparing the pharmaceutical compositions can be non-toxic in the amounts used. It will be evident to those of ordinary skill in the art that the optimal dosage of the active ingredient(s) in the pharmaceutical composition will depend on a variety of factors. Relevant factors include, without limitation, the type of subject (e.g., human), the overall health of the subject, the type of cancer the subject is in need of treatment of, the use of the composition as part of a multi-drug regimen, the particular form of the compound of the invention, the manner of administration, and the composition employed.

The pharmaceutically acceptable carrier or vehicle may be particulate, so that the compositions are, for example, in tablet or powder form. The carrier(s) can be liquid, with the compositions being, for example, an oral syrup or injectable liquid. In addition, the carrier(s) can be gaseous, so as to provide an aerosol composition useful in, e.g., inhalatory administration.

The composition may be intended for oral administration, and if so, the composition is preferably in solid or liquid form, where semi-solid, semi-liquid, suspension and gel forms are included within the forms considered herein as either solid or liquid.

As a solid composition for oral administration, the composition can be formulated into a powder, granule, compressed tablet, pill, capsule, chewing gum, wafer or the like form. Such a solid composition typically contains one or more inert diluents. In addition, one or more of the following can be present: binders such as ethyl cellulose, carboxymethylcellulose, microcrystalline cellulose, or gelatin; excipients such as starch, lactose or dextrins, disintegrating agents such as alginic acid, sodium alginate, Primogel, corn starch and the like; lubricants such as magnesium stearate or Sterotex; glidants such as colloidal silicon dioxide; sweetening agents such as sucrose or saccharin, a flavoring agent such as peppermint, methyl salicylate or orange flavoring, and a coloring agent.

When the pharmaceutical composition is in the form of a capsule, e.g., a gelatin capsule, it can contain, in addition to materials of the above type, a liquid carrier such as polyethylene glycol, cyclodextrin or a fatty oil.

The pharmaceutical composition can be in the form of a liquid, e.g., an elixir, syrup, solution, emulsion or suspension. The liquid can be useful for oral administration or for delivery by injection. When intended for oral administration, a composition can comprise one or more of a sweetening agent, preservatives, dye/colorant and flavor enhancer. In a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is a preferred adjuvant. An injectable composition is preferably sterile.

The amount of the compound of the invention that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

The pharmaceutical compositions comprise an effective amount of a compound of the invention such that a suitable dosage will be obtained. Typically, this amount is at least 0.01% of a compound of the invention by weight of the composition. When intended for oral administration, this amount can be varied to be between 0.1% and 80% by weight of the composition. Preferred oral compositions can comprise from between 4% and 50% of the compound of the invention by weight of the composition. Preferred compositions of the present invention are prepared so that a parenteral dosage unit contains from between 0.01% and 2% by weight of the compound of the invention.

The compounds of the invention can be administered in a single dose or in multiple doses.

In one embodiment, the compounds of the invention are administered in multiple doses. When administered in multiple doses, the compounds are administered with a frequency and in an amount sufficient to treat the condition. In one embodiment, the frequency of administration ranges from once a day up to about once every eight weeks. In another embodiment, the frequency of administration ranges from about once a week up to about once every six weeks. In another embodiment, the frequency of administration ranges from about once every three weeks up to about once every four weeks.

Preliminary toxicological studies suggest that the compounds of the invention can be administered to a subject in an amount similar to, or less than, that used with other platinum cytotoxic agents such as cis-bis-neodecanoato-trans-RR-1,2-dicyclohexane platinum (II), (NDDP) or cisplatin. Generally, the dosage of a compound of the invention administered to a subject is in the range of 0.1 to 150 mg/kg, and more typically, in the range of 0.1 mg/kg to 100 mg/kg, of the subject's body weight. In one embodiment, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the subject's body weight.

The compounds of the invention can be administered by any convenient route, for example by infusion or bolus injection, by absorption through epithelial or mucocutaneous linings (e.g., oral mucosa, rectal and intestinal mucosa, etc.). Administration can be systemic or local. Various delivery systems are known, e.g., microparticles, microcapsules, capsules, etc., and may be useful for administering a compound of the invention. In certain embodiments, more than one compound of the invention is administered to a subject. Methods of administration may include, but are not limited to, oral administration and parenteral administration; parenteral administration including, but not limited to, intradermal, intramuscular, intraperitoneal, intravenous, subcutaneous; intranasal, epidural, sublingual, intranasal, intracerebral, intraventricular, intrathecal, intravaginal, transdermal, rectally, by inhalation, or topically to the ears, nose, eyes, or skin. The preferred mode of administration is left to the discretion of the practitioner, and will depend in-part upon the site of the medical condition (such as the site of cancer, a cancerous tumor or a pre-cancerous condition).

In one embodiment, the compounds of the invention are administered parenterally.

In a preferred embodiment, the compounds of the invention are administered intravenously.

In specific embodiments, it can be desirable to administer one or more compounds of the invention locally to the area in need of treatment. This can be achieved, for example, and not by way of limitation, by local infusion during surgery; topical application, e.g., in conjunction with a wound dressing after surgery; by injection; by means of a catheter; by means of a suppository; or by means of an implant, the implant being of a porous, non-porous, or gelatinous material, including membranes, such as sialastic membranes, or fibers. In one embodiment, administration can be by direct injection at the site (or former site) of a cancer, tumor, or precancerous tissue. In certain embodiments, it can be desirable to introduce one or more compounds of the invention into the central nervous system by any suitable route, including intraventricular and intrathecal injection. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the compounds of the invention can be formulated as a suppository, with traditional binders and carriers such as triglycerides.

In yet another embodiment, the compounds of the invention can be delivered in a controlled release system. In one embodiment, a pump can be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, J. Macromol. Sci. Rev. Macromol. Chem. 23:61 (1983); see also Levy et al., Science 228:190 (1985); During et al., Ann. Neurol. 25:351 (1989); Howard et al., J. Neurosurg. 71:105 (1989)). In yet another embodiment, a controlled-release system can be placed in proximity of the target of the compounds of the invention, e.g., the brain, thus requiring only a fraction of the systemic dose (see, e.g., Goodson, in Medical Applications of Controlled Release, supra, vol. 2, pp. 115-138 (1984)). Other controlled-release systems discussed in the review by Langer (Science 249:1527-1533 (1990)) can be used.

The term “carrier” refers to a diluent, adjuvant or excipient, with which a compound of the invention is administered. Such pharmaceutical carriers can be liquids, such as water and oils, including those of petroleum, animal, vegetable or synthetic origin, such as peanut oil, soybean oil, mineral oil, sesame oil and the like. The carriers can be saline, gum acacia, gelatin, starch paste, talc, keratin, colloidal silica, urea, and the like. In addition, auxiliary, stabilizing, thickening, lubricating and coloring agents can be used. In one embodiment, when administered to a subject, the compounds of the invention and pharmaceutically acceptable carriers are sterile. Water is a preferred carrier when the compound of the invention is administered intravenously. Saline solutions and aqueous dextrose and glycerol solutions can also be employed as liquid carriers, particularly for injectable solutions. Suitable pharmaceutical carriers also include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable carrier is a capsule (see e.g., U.S. Pat. No. 5,698,155). Other examples of suitable pharmaceutical carriers are described in “Remington's Pharmaceutical Sciences” by E.W. Martin.

Sustained or directed release compositions that can be formulated include, but are not limited to, the liposomal lipid platinum complexes of the invention and other formulations where a lipid platinum complex of the invention is protected with differentially degradable coatings, e.g., by microencapsulation, multiple coatings, etc. It is also possible to freeze-dry the compositions and use the lyophilizates obtained, for example, for the preparation of products for injection.

In a preferred embodiment, the lipid platinum complexes of the invention are encapsulated in a liposome. In a particularly preferred embodiment, the liposome is less than 1 μM in diameter.

In a preferred embodiment, the compounds of the invention are formulated in accordance with routine procedures as a pharmaceutical composition adapted for intravenous administration to animals, particularly human beings. Typically, the carriers or vehicles for intravenous administration are sterile isotonic aqueous buffer solutions. Where necessary, the compositions can also include a solubilizing agent. Compositions for intravenous administration can optionally comprise a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where a compound of the invention is to be administered by infusion, it can be dispensed, for example, with an infusion bottle containing sterile pharmaceutical grade water or saline. Where the compound of the invention is administered by injection, an ampoule of sterile water for injection or saline can be provided so that the ingredients can be mixed prior to administration.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving complex are also suitable for orally administered compositions of the invention. In these later platforms, fluid from the environment surrounding the capsule is imbibed by the driving complex, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard carriers such as mannitol, lactose, starch, magnesium stearate, sodium saccharine, cellulose, magnesium carbonate, etc. Such carriers are preferably of pharmaceutical grade.

The pharmaceutical compositions of the invention can be intended for topical administration, in which case the carrier can be in the form of a solution, emulsion, ointment or gel base. The base, for example, can comprise one or more of the following: petrolatum, lanolin, polyethylene glycols, beeswax, mineral oil, diluents such as water and alcohol, and emulsifiers and stabilizers. Thickening agents can be present in a composition for topical administration. If intended for transdermal administration, the composition can be in the form of a transdermal patch or an iontophoresis device. Topical formulations can comprise a concentration of a compound of the invention of from between 0.01% and 10% w/v (weight per unit volume of composition).

The compositions can include various materials that modify the physical form of a solid or liquid dosage unit. For example, the composition can include materials that form a coating shell around the active ingredients. The materials that form the coating shell are typically inert, and can be selected from, for example, sugar, shellac, and other enteric coating agents. Alternatively, the active ingredients can be encased in a gelatin capsule.

The compositions can consist of gaseous dosage units, e.g., it can be in the form of an aerosol. The term aerosol is used to denote a variety of systems ranging from those of colloidal nature to systems consisting of pressurized packages. Delivery can be by a liquefied or compressed gas or by a suitable pump system that dispenses the active ingredients. Aerosols of the compositions can be delivered in single phase, bi-phasic, or tri-phasic systems in order to deliver the composition. Delivery of the aerosol includes the necessary container, activators, valves, subcontainers, Spacers and the like, which together can form a kit. Preferred aerosols can be determined by one skilled in the art, without undue experimentation.

Whether in solid, liquid or gaseous form, the compositions of the present invention can comprise an additional therapeutically active agent selected from among those including, but not limited to, an additional anticancer agent, an antiemetic agent, a hematopoietic colony stimulating factor, an anti-depressant and an analgesic agent.

The pharmaceutical compositions can be prepared using methodology well known in the pharmaceutical art. For example, a composition intended to be administered by injection can be prepared by combining a compound of the invention with water so as to form a solution. A surfactant can be added to facilitate the formation of a homogeneous solution or suspension. Surfactants are complexes that can non-covalently interact with a compound of the invention so as to facilitate dissolution or homogeneous suspension of the compound of the invention in the aqueous delivery system.

In one embodiment, the pharmaceutical compositions of the present invention may comprise one or more known therapeutically active agents.

In another embodiment, the pharmaceutical compositions of the present invention can be administered prior to, at the same time as, or after an additional anticancer agent, or on the same day, or within 1 hour, 2 hours, 12 hours, 24 hours, 48 hours, 72 hours, 1 week, 2 weeks, 3 weeks or 4 weeks of each other.

In another embodiment, the pharmaceutical compositions of the present invention can be administered prior to, at the same time as, or after an antiemetic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after a hematopoietic colony stimulating factor, or on the same day, or within 1 hour, 2 hours, 12 hours, 24 hours, 48 hours, 72 hours, 1 week, 2 weeks, 3 weeks or 4 weeks of each other.

In another embodiment, the pharmaceutical compositions of the present invention can be administered prior to, at the same time as, or after an opioid or non-opioid analgesic agent, or on the same day, or within 1 hour, 2 hours, 12 hours, 24 hours, 48 hours or 72 hours of each other.

In another embodiment, the pharmaceutical compositions of the present invention can be administered prior to, at the same time as, or after an anti-depressant agent, or on the same day, or within 1 hour, 2 hours, 12 hours, 24 hours, 48 hours or 72 hours of each other.

### 4.5.1 Kits

The invention encompasses kits that can simplify the administration of a compound or composition of the invention to a subject.

A typical kit of the invention comprises a unit dosage of a compound of the invention. In one embodiment, the unit dosage form is in a container, which can be sterile, containing an effective amount of a compound of the invention and a pharmaceutically acceptable carrier or vehicle. In another embodiment, the unit dosage form is in a container containing an effective amount of a compound of the invention as a lyophilate. In this instance, the kit can further comprise a second container which contains a solution useful for the reconstitution of the lyophiliate, such as saline or phosphate buffered saline. The kit can also comprise a label or printed instructions for use of a compound of the invention. The kit can further comprise a unit dosage form of another therapeutically active agent. In one embodiment, the kit comprises a container containing an amount of an additional anticancer agent effective to treat cancer. In another embodiment the kit comprises a container containing a therapeutically active agent such as an antiemetic agent, a hematopoietic colony-stimulating factor, an analgesic agent or an anxiolytic agent.

In one embodiment, the kit comprises a unit dosage form of a pharmaceutical composition of the invention.

Kits of the invention can further comprise a device that is useful for administering the unit dosage forms of a compound or pharmaceutical composition of the invention. Examples of such devices include, but are not limited to, a syringe, a drip bag, a patch or an enema, which optionally contain the unit dosage forms.

### 4.6 Therapeutic Uses

### 4.6.1 Treatment Of Cancer

Cancer or a neoplastic disease, including, but not limited to, neoplasms, tumors, metastases, or any disease or disorder characterized by uncontrolled cell growth, can be treated, suppressed, delayed, inhibited or prevented by administration of an amount of a compound of the invention effective to treat cancer or by administration of an amount of a composition effective to treat cancer, said composition comprising a pharmaceutically acceptable carrier and a compound of the invention. When the compound of the invention is a lipid platinum complex, the compositions can comprise a pharmaceutically acceptable salt thereof. The invention as it applies to cancer encompasses the treatment, suppression, delaying, inhibiting of growth and/or progression, and prevention of cancer or neoplastic disease as described herein.

### 4.6.1.1 Therapeutic Methods

In a preferred embodiment, the present invention provides methods for treating cancer, including: killing a cancer cell or neoplastic cell; inhibiting the growth of a cancer cell or neoplastic cell; inhibiting the replication of a cancer cell or neoplastic cell; or ameliorating a symptom thereof, said methods comprising administering to a subject in need thereof an amount of a compound of the invention effective to treat cancer.

The compounds of the invention can be used accordingly in a variety of settings for the treatment of various cancers. Without being bound by theory, in one embodiment, a lipid platinum complex of the invention can enter a cell by diffusion and react with DNA to form interstrand and intrastrand cross-links and DNA-protein crosslinks, which can interfere with the ability of the cell to replicate.

In a specific embodiment, the subject in need of treatment has previously undergone treatment for cancer. Such previous treatments include, but are not limited to, prior chemotherapy, radiation therapy, surgery or immunotherapy, such as cancer vaccines.

In another embodiment, the cancer being treated is a cancer which has demonstrated sensitivity to platinum therapy or is known to be responsive to platinum therapy. Such cancers include, but are not limited to, small-cell lung cancer, non-small cell lung cancer, ovarian cancer, breast cancer, bladder cancer, testicular cancer, head and neck cancer, colorectal cancer, Hodgkin's disease, leukemia, osteogenic sarcoma, and melanoma.

In still another embodiment, the cancer being treated is a cancer which has demonstrated resistance to platinum therapy or is known to be refractory to platinum therapy. Such refractory cancers include, but are not limited to, cancers of the cervix, prostate, and esophagus. A cancer may be determined to be refractory to a therapy when at least some significant portion of the cancer cells are not killed or their cell division are not arrested in response to the therapy. Such a determination can be made either in vivo or in vitro by any method known in the art for assaying the effectiveness of treatment on cancer cells, using the art-accepted meanings of “refractory” in such a context. In a specific embodiment, a cancer is refractory where the number of cancer cells has not been significantly reduced, or has increased. Such cancers include, but are not limited to, cancers of the cervix, prostate, and esophagus.

Other cancers that can be treated with the compounds of the invention include, but are not limited to, cancers disclosed below in Table 1 and metastases thereof.

TABLE 1

Solid tumors, including but not limited to:
fibrosarcoma
myxosarcoma
liposarcoma
chondrosarcoma
osteogenic sarcoma
chordoma
angiosarcoma
endotheliosarcoma
lymphangiosarcoma
lymphangioendotheliosarcoma
synovioma
mesothelioma
Ewing's tumor
leiomyosarcoma
rhabdomyosarcoma
colon cancer
colorectal cancer
kidney cancer
pancreatic cancer
bone cancer
breast cancer
ovarian cancer
prostate cancer
esophageal cancer
stomach cancer
oral cancer
nasal cancer
throat cancer
squamous cell carcinoma
basal cell carcinoma
adenocarcinoma
sweat gland carcinoma
sebaceous gland carcinoma
papillary carcinoma
papillary adenocarcinomas
cystadenocarcinoma
medullary carcinoma
bronchogenic carcinoma
renal cell carcinoma
hepatoma
bile duct carcinoma
choriocarcinoma
seminoma
embryonal carcinoma
Wilms' tumor
cervical cancer
uterine cancer
testicular cancer
small cell lung carcinoma
bladder carcinoma
lung cancer
epitheial carcinoma
glioma
glioblastoma multiforme
astrocytoma
medulloblastoma
craniopharyngioma
ependymoma
pinealoma
hemangioblastoma
acoustic neuroma
oligodendroglioma
meningioma
skin cancer
melanoma
neuroblastoma
retinoblastoma
blood-borne cancers, including but not limited to:
acute lymphoblastic leukemia “ALL”
acute lymphoblastic B-cell leukemia
acute lymphoblastic T-cell leukemia
acute myeloblastic leukemia “AML”
acute promyelocytic leukemia “APL”
acute monoblastic leukemia
acute erytbioleukemic leukemia
acute megakaryoblastic leukemia
acute myelomonocytic leukemia
acute nonlymphocyctic leukemia
acute undifferentiated leukemia
chronic myelocytic leukemia “CML”
chronic lymphocytic leukemia “CLL”
hairy cell leukemia
multiple myeloma
acute and chronic leukemias:
lymphoblastic
myclogenous
lymphocytic
myelocytic leukemias
Lymphomas:
Hodgkin's disease
non-Hodgkin's Lymphoma
Multiple myeloma
Waldenström's macroglobulinemia
Heavy chain disease
Polycythemia vera

In one embodiment, the cancer is selected from the group consisting of pancreatic cancer, colorectal cancer, mesothelioma, a malignant pleural effusion, peritoneal carcinomatosis, peritoneal sarcomatosis, renal cell carcinoma, small cell lung cancer, non-small cell lung cancer, testicular cancer, bladder cancer, breast cancer, head and neck cancer, and ovarian cancer.

In a preferred embodiment the cancer is pancreatic cancer, colorectal cancer or mesothelioma.

### 4.6.1.2 Prophylactic Methods

The compounds of the invention can also be administered to prevent progression to a neoplastic or malignant state, including but not limited to the cancers listed in Table 1. Such prophylactic use is indicated in conditions known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell, 1976, Basic Pathology, 2d Ed., W.B. Saunders Co., Philadelphia, pp. 68-79.). Hyperplasia is a form of controlled cell proliferation involving an increase in cell number in a tissue or organ, without significant alteration in structure or function. For example, endometrial hyperplasia often precedes endometrial cancer and precancerous colon polyps often transform into cancerous lesions. Metaplasia is a form of controlled cell growth in which one type of adult or fully differentiated cell substitutes for another type of adult cell. Metaplasia can occur in epithelial or connective tissue cells. A typical metaplasia involves a somewhat disorderly metaplastic epithelium. Dysplasia is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation, and is often found in the cervix, respiratory passages, oral cavity, and gall bladder.

Alternatively or in addition to the presence of abnormal cell growth characterized as hyperplasia, metaplasia, or dysplasia, the presence of one or more characteristics of a transformed phenotype, or of a malignant phenotype, displayed in vivo or displayed in vitro by a cell sample from a patient, can indicate the desirability of prophylactic/therapeutic administration of the composition of the invention. Such characteristics of a transformed phenotype include morphology changes, looser substratum attachment, loss of contact inhibition, loss of anchorage dependence, protease release, increased sugar transport, decreased serum requirement, expression of fetal antigens, disappearance of the 250,000 dalton cell surface protein, etc. (see also id., at pp. 84-90 for characteristics associated with a transformed or malignant phenotype).

In a specific embodiment, leukoplakia, a benign-appearing hyperplastic or dysplastic lesion of the epithelium, or Bowen's disease, a carcinoma in situ, are pre-neoplastic lesions indicative of the desirability of prophylactic intervention.

In another embodiment, fibrocystic disease (cystic hyperplasia, mammary dysplasia, particularly adenosis (benign epithelial hyperplasia)) is indicative of the desirability of prophylactic intervention.

The prophylactic use of the compounds of the invention is also indicated in some viral infections that may lead to cancer. For example, human papilloma virus can lead to cervical cancer (see, e.g., Hernandez-Avila et al., Archives of Medical Research (1997) 28:265-271), Epstein-Barr virus (EBV) can lead to lymphoma (see, e.g., Herrmann et al., J Pathol (2003) 199(2):140-5), hepatitis B or C virus can lead to liver carcinoma (see, e.g., El-Serag, J Clin Gastroenterol (2002) 35(5 Suppl 2):S72-8), human T cell leukemia virus (HTLV)-I can lead to T-cell leukemia (see e.g., Mortreux et al., Leukemia (2003) 17(1):26-38), human herpesvirus-8 infection can lead to Kaposi's sarcoma (see, e.g., Kadow et al., Curr. Opin. Investig. Drugs (2002) 3(11):1574-9), and Human Immune deficiency Virus (HIV) infection contribute to cancer development as a consequence of immunodeficiency (see, e.g., Dal Maso et al., Lancet Oncol. (2003) 4(2):110-9).

In other embodiments, a patient which exhibits one or more of the following predisposing factors for malignancy can treated by administration of an effective amount of a compound of the invention: a chromosomal translocation associated with a malignancy (e.g., the Philadelphia chromosome for chronic myelogenous leukemia, t(14; 18) for follicular lymphoma, etc.), familial polyposis or Gardner's syndrome (possible forerunners of colon cancer), benign monoclonal gammopathy (a possible forerunner of multiple myeloma), a first degree kinship with persons having a cancer or precancerous disease showing a Mendelian (genetic) inheritance pattern (e.g., familial polyposis of the colon, Gardner's syndrome, hereditary exostosis, polyendocrine adenomatosis, medullary thyroid carcinoma with amyloid production and pheochromocytoma, Peutz-Jeghers syndrome, neurofibromatosis of Von Recklinghausen, retinoblastoma, carotid body tumor, cutaneous melanocarcinoma, intraocular melanocarcinoma, xeroderma pigmentosum, ataxia telangiectasia, Chediak-Higashi syndrome, albinism, Fanconi's aplastic anemia, and Bloom's syndrome; see Robbins and Angell, 1976, Basic Pathology, 2d Ed., W.B. Saunders Co., Philadelphia, pp. 112-113) etc.), and exposure to carcinogens (e.g., smoking, and inhalation of or contacting with certain chemicals).

In another specific embodiment, a composition of the invention is administered to a human patient to prevent, delay or inhibit the growth and/or progression of breast, colon, ovarian, or cervical cancer.

In another specific embodiment, a composition of the invention is administered to a human patient to delay progression to breast, colon, ovarian, or cervical cancer.

### 4.6.2 Multi-Modality Therapy for Cancer

The compounds of the invention can be administered to a subject that has undergone or is currently undergoing one or more additional anticancer treatment modalities including, but not limited to, surgery, radiation therapy, or immunotherapy, such as cancer vaccines.

In one embodiment, the invention provides methods for treating cancer comprising (a) administering to a subject in need thereof an amount of a compound of the invention effective to treat cancer; and (b) administering to said subject one or more additional anticancer treatment modalities including, but not limited to, surgery, radiation therapy, or immunotherapy, such as a cancer vaccine.

In one embodiment, the additional anticancer treatment modality is radiation therapy.

In another embodiment, the additional anticancer treatment modality is surgery.

In still another embodiment, the additional anticancer treatment modality is immunotherapy.

In a specific embodiment, the compound of the invention is administered concurrently with radiation therapy. In another specific embodiment, the additional anticancer treatment modality is administered prior or subsequent to administration of a compound of the invention, preferably at least an hour, five hours, 12 hours, a day, a week, a month, more preferably several months (e.g., up to three months), prior or subsequent to administration of a compound of the invention.

When the additional anticancer treatment modality is radiation therapy, any radiation therapy protocol can be used depending upon the type of cancer to be treated. For example, but not by way of limitation, x-ray radiation can be administered; in particular, high-energy megavoltage (radiation of greater that 1 MeV energy) can be used for deep tumors, and electron beam and orthovoltage x-ray radiation can be used for skin cancers. Gamma-ray emitting radioisotopes, such as radioactive isotopes of radium, cobalt and other elements, can also be administered.

Additionally, the invention provides methods of treatment of cancer with a compound of the invention as an alternative to chemotherapy or radiation therapy where the chemotherapy or the radiation therapy has proven or can prove too toxic, e.g., results in unacceptable or unbearable side effects, for the subject being treated. The subject being treated can, optionally, be treated with another anticancer treatment modality such as surgery, radiation therapy or immunotherapy, depending on which treatment is found to be acceptable or bearable.

The compounds of the invention can also be used in an in vitro or ex vivo fashion, such as for the treatment of certain cancers, including, but not limited to leukemias and lymphomas, such treatment involving autologous stem cell transplants. This can involve a multi-step process in which the animal's autologous hematopoietic stem cells are harvested and purged of all cancer cells, the patient's remaining bone-marrow cell population is then eradicated via the administration of a high dose of a compound of the invention with or without additional anticancer agents and/or high dose radiation therapy, and the stem cell graft is infused back into the animal. Supportive care is then provided while bone marrow function is restored and the subject recovers.

### 4.6.3 Multi-Drug Therapy for Cancer

The present invention also provides methods for treating cancer comprising administering to a subject in need thereof an amount of a compound of the invention effective to treat cancer and one or more additional anticancer agents or pharmaceutically acceptable salts thereof, said additional anticancer agents not being compounds of the invention. The combination of agents can act additively or synergistically. Suitable additional anticancer agents include, but are not limited to, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idaribicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

In one embodiment, the additional anticancer agent can be, but is not limited to, a drug listed in Table 2.

TABLE 2

| | |
|---|---|
| Alkylating agents | |
| Nitrogen mustards: | Cyclophosphamide |
| | Ifosfamide |
| | Trofosfamide |
| | Chlorambucil |
| Nitrosoureas: | Carmustine (BCNU) |
| | Lomustine (CCNU) |
| Alkylsuiphonates: | Busulfan |
| | Treosulfan |
| Triazenes: | Dacarbazine |
| Platinum containing complexes: | Cisplatin |
| Carboplatin | |
| Aroplatin | |
| Oxaliplatin | |
| Plant Alkaloids | |
| Vinca alkaloids: | Vincristine |
| | Vinblastine |
| | Vindesine |
| | Vinorelbine |
| Taxoids: | Pacitaxel |
| | Docetaxel |
| DNA Topoisomerase Inhibitors | |
| Epipodophyllins: | Etoposide |
| | Teniposide |
| | Topotecan |
| | 9-aminocamptothecin |
| | Camptothecin |
| | Crisnatol |
| Mitomycins: | Mitomycin C |
| | Anti-metabolites |
| Anti-folates: | |
| DHFR inhibitors: | Methotrexate |
| | Trimetrexate |
| IMP dehydrogenase Inhibitors: | Mycophenolic acid |
| | Tiazofurin |
| | Ribavirin |
| | EICAR |
| Ribonuclotide reductase | Hydroxyurea |
| Inhibitors: | |
| | Deferoxamine |
| Pyrimidine analogs: | |
| Uracil analogs: | 5-Fluorouracil |
| | Floxuridine |
| | Doxifluridine |
| | Ratitrexed |
| Cytosine analogs: | Cytarabine (ara C) |
| | Cytosine arabinoside |
| | Fludarabine |
| | Gemcitabine |
| | Capecitabine |
| Purine analogs: | Mercaptopurine |
| | Thioguanine |
| DNA Antimetabolites: | 3-HP |
| | 2′-deoxy-5-fluorouridine |
| | 5-HP |
| | alpha-TGDR |
| | aphidicolin glycinate |
| | ara-C |
| | 5-aza-2′-deoxycytidine |
| | beta-TGDR |
| | cyclocytidine |
| | guanazole |
| | inosine glycodialdehyde |
| | macebecin II |
| | Pyrazoloimidazole |
| Hormonal therapies: | |
| Receptor antagonists: | |
| Anti-estrogen: | Tamoxifen |
| | Raloxifene |
| | Megestrol |
| LHRH agonists: | Goscrclin |
| | Leuprolide acetate |
| Anti-androgens: | Flutamide |
| | Bicalutamide |
| Retinoids/Deltoids | |
| | Cis-retinoic acid |
| Vitamin A derivative: | All-trans retinoic acid (ATRA-IV) |
| Vitamin D3 analogs: | EB 1089 |
| | CB 1093 |
| | KB 1060 |
| Photodynamic therapies: | Vertoporfin (BPD-MA) |
| | Phthalocyanine |
| | Photosensitizer Pc4 |
| | Demethoxy-hypocrellin A |
| | (2BA-2-DMHA) |
| Cytokines: | Interferon-α |
| | Interferon-β |
| | Interferon-γ |
| | Tumor necrosis factor |
| Angiogenesis Inhibitors: | Angiostatin (plasminogen |
| | fragment) |
| | antiangiogenic antithrombin III |
| | Angiozyme |
| | ABT-627 |
| | Bay 12-9566 |
| | Benefin |
| | Bevacizumab |
| | BMS-275291 |
| | cartilage-derived inhibitor (CDI) |
| | CAI |
| | CD59 complement fragment |
| | CEP -7055 |
| | Col 3 |
| | Combretastatin A-4 |
| | Endostatin (collagen XVIII |
| | fragment) |
| | Fibronectin fragment |
| | Gro-beta |
| | Halofuginone |
| | Heparinases |
| | Heparin hexasaccharide fragment |
| | HMV833 |
| | Human chorionic gonadotropin |
| | (hCG) |
| | IM-862 |
| | Interferon alpha/beta/gamma |
| | Interferon inducible protein (IP- |
| | 10) |
| | Interleukin-12 |
| | Kringle 5 (plasminogen fragment) |
| | Marimastat |
| | Metalloproteinase inhibitors |
| | (TIMPs) |
| | 2-Methoxyestradiol |
| | MMI 270 (CGS 27023A) |
| | MoAb IMC-1C11 |
| | Neovastat |
| | NM-3 |
| | Panzem |
| | PI-88 |
| | Placental ribonuclease inhibitor |
| | Plasminogen activator inhibitor |
| | Platelet factor-4 (PF4) |
| | Prinomastat |
| | Prolactin 16kD fragment |
| | Proliferin-related protein (PRP) |
| | PTK 787/ZK 222594 |
| | Retinoids |
| | Solimastat |
| | Squalamine |
| | SS 3304 |
| | SU 5416 |
| | SU6668 |
| | SU11248 |
| | Tetrahydrocortisol-S |
| | Tetrathiomolybdate |
| | Thalidomide |
| | Thrombospondin-1 (TSP-1) |
| | TNP-470 |
| | Transforming growth factor-beta |
| | (TGF-b) |
| | Vasculostatin |
| | Vasostatin (calreticulin fragment) |
| | ZD6126 |
| | ZD 6474 |
| | farnesyl transferase inhibitors |
| | (FTI) |
| | Bisphosphonates |
| Antimitotic agents: | Allocolchicine |
| | Halichondrin B |
| | Colchicine |
| | colchicine derivative |
| | dolstatin 10 |
| | Maytansine |
| | Rhizoxin |
| | Thiocolchicine |
| | trityl cysteine |
| Others: | |
| Isoprenylation inhibitors: | |
| Dopaminergic neurotoxins: | 1-methyl-4-phenylpyridinium ion |
| Cell cycle inhibitors: | Staurosporine |
| Actinomycins: | Actinomycin D |
| | Dactinomycin |
| Bleomycins: | Bleomycin A2 |
| | Bleomycin B2 |
| | Peplomycin |
| Anthracyclines: | Daunorubicin |
| | Doxorubicin (adriamycin) |
| | Idarubicin |
| | Epirubicin |
| | Pirarubicin |
| | Zorubicin |
| | Mitoxantrone |
| MDR inhibitors: | Verapamil |
| Ca²⁺ ATPase inhibitors: | Thapsigargin |

Additional anticancer agents that can be used in the compositions and methods of the present invention include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine hydrochloride; hydroxyurea; idarubicin hydrochloride; ifosfamide; ilmofosine; interleukin II (including recombinant interleukin II, or rIL2), interferon alfa-2α; interferon alfa-2β; interferon alfa-n1; interferon alfa-n3; interferon beta-Iα; interferon gamma-Iβ; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anticancer drugs that can be used include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulnic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-acytidine; dihydrotaxol; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum complexes; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agents; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor, platinum complex; platinum complexes; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, micro algal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone Bl; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor, stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

In a preferred embodiment, the additional anticancer agent is gemcitabine, capecitabine or 5-fluorouracil.

### 4.7 Other Therapeutic Agents

The present methods can further comprise the administration of a compound of the invention and another therapeutically active agent or pharmaceutically acceptable salt thereof. The compound of the invention and the therapeutically active agent can act additively or, more preferably, synergistically. In a preferred embodiment, a compound of the invention is administered concurrently with the administration of one or more other therapeutically active agents, which can be part of the same composition or in a different composition from that comprising the compound of the invention. In another embodiment, a compound of the invention is administered prior to or subsequent to administration of one or more other therapeutically active agents. Kits comprising a compound of the invention, preferably purified, and one or more therapeutically active agents, in one or more containers are also provided.

In the present methods for treating cancer the other therapeutically active agent can be an antiemetic agent. Suitable antiemetic agents include, but are not limited to, metoclopromide, domperidone, prochlorperazine, promethazine, chlorpromazine, trimethobenzamide, ondansetron, granisetron, hydroxyzine, acethylleucine monoethanolamine, alizapride, azasetron, benzquinamide, bietanautine, bromopride, buclizine, clebopride, cyclizine, dimenhydrinate, diphenidol, dolasetron, meclizine, methallatal, metopimazine, nabilone, oxyperndyl, pipamazine, scopolamine, sulpiride, tetrahydrocannabinols, thiethylperazine, thioproperazine and tropisetron.

In a preferred embodiment, the antiemetic agent is granisetron or ondansetron.

In another embodiment, the other therapeutically active agent can be an hematopoietic colony stimulating factor. Suitable hematopoietic colony stimulating factors include, but are not limited to, filgrastim, sargramostim, molgramostim and epoietin alfa.

In still another embodiment, the other therapeutically active agent can be an opioid or non-opioid analgesic agent. Suitable opioid analgesic agents include, but are not limited to, morphine, heroin, hydromorphone, hydrocodone, oxymorphone, oxycodone, metopon, apomorphine, normorphine, etorphine, buprenorphine, meperidine, lopermide, anileridine, ethoheptazine, piminidine, betaprodine, diphenoxylate, fentanil, sufentanil, alfentanil, remifentanil, levorphanol, dextromethorphan, phenazocine, pentazocine, cyclazocine, methadone, isomethadone and propoxyphene. Suitable non-opioid analgesic agents include, but are not limited to, aspirin, celecoxib, rofecoxib, diclofinac, diflusinal, etodolac, fenoprofen, flurbiprofen, ibuprofen, ketoprofen, indomethacin, ketorolac, meclofenamate, mefanamic acid, nabumetone, naproxen, piroxicam and sulindac.

In yet another embodiment, the other therapeutically active agent can be an anxiolytic agent. Suitable anxiolytic agents include, but are not limited to, buspirone, and benzodiazepines such as diazepam, lorazepam, oxazapam, chlorazepate, clonazepam, chlordiazepoxide and alprazolam.

### 5. EXAMPLES

### 5.1 Example 1

### Synthesis Cis-[Trans-(1R,2R)-1,2-Diamino-Cyclohexane]Diodoplatinum(II), (10)

A filtered solution of K₂PtCl₄ (1251.0 g, 3.014 mol) (Alfa Aesar, Ward Hill, Mass.) in water (10 L) was added to a solution of KI (2919 g, 17.6 mol) in water (5 L) at 25° C. and stirred for 10 min. To the resultant solution was slowly added a solution of trans-(1R,2R)-1,2-diaminecyclohexane (364.0 g, 3.188 mol) (11) (Alfa Aesar) in deionized water (1.3 L). The reaction mixture was stirred for 3 hours at 25° C., filtered, and the resultant yellow precipitate washed with 3×2 L of deionized water. The precipitate was suspended in 8 L of deionized water, filtered, and the solids washed with 6×1.5 L of deionized water at which point the filtrate showed no positive reaction towards AgNO₃. The solids were re-suspended in dimethylformamide (DMF) (1.5 L) and filtered. The filter cake was then washed with DMF (0.3 L), water (3×1 L) and acetone (3×0.7 L). The solids were collected and dried under reduced pressure to provide cis-[trans-(1R,2R)-1,2-diaminocyclohexane]diiodoplatinum(II) (10) as a light yellow powder (1527.6 g, 90%).

### 5.2 Example 2

### Synthesis of 1,2-Dimyristoyl-Sn-Glycero-3-Phospho(Rac-1-Glycerol, Silver Salt, (12)

A solution of silver nitrate (0.30 g, 1.75 mmol, 2.0 eq.) in 3:2 (v:v) water/ethanol (15 mL) was added to a stirred suspension of 1,2-dimyristoyl-sn-glycero-3-phospho(rac-1-glycerol), sodium salt (13) (1.21 g, 1.75 mmol) (Lipoid, GMBH, Ludwigshafen, Germany) in a 3:2 water/ethanol mixture (50 mL) at 25° C. The resultant reaction mixture was protected from light and stirred for 24 hours at 25° C. The mixture was filtered, and the resultant white solids were washed with deionized water (3×25 mL) and dried under reduced pressure to provide 1,2-dimyristoyl-sn-glycero-3-phospho(rac-1-glycerol), silver salt, (12) (1.11 g; 81%).

### 5.3 Example 3

### Synthesis of Cis-Bis[1,2-Dimyristoyl-Sn-Glycero-3-Phospho(Rac-1-Glycerol)]Trans(1R,2R)-1,2-Diaminocyclohexane]Platinum(II), 14

A suspension of complex 10 (0.81 g, 1.44 mmol, 2.0 eq.) in chloroform (100 mL) was added to a stirred solution of 12 (1.11 g, 1.43 mmol) in chloroform (50 mL) at 25° C. The resultant suspension was protected from light, ultrasonicated for 2 hours, and stirred for 15 hours at 25° C. The resultant mixture was filtered and the solids washed with chloroform (3×40 mL). The filtrates were combined and the solvent removed under reduced pressure. The resultant oily residue was crystallized from acetone, re-crystallized from a chloroform-acetone mixture, and the resulting precipitate was dried under reduced pressure to provide cis-bis[1,2-dimyristoyl-sn-glycero-3-phospho(rac-1-glycerol)][trans-(1R,2R)-1,2-diaminocyclohexane]platinum(II), (14) (0.96 g, 41%). The purity and structure of platinum complex 14 was confirmed by elemental analysis and mass spectroscopy.

Complex 14: Anal. Calcd. for C74H146N2O20P2Pt×H₂O: C 53.57; H, 8.99, N 1.69, P 3.73, Pt 11.76; Found: C 53.09; H, 9.01, N 1.69, P 3.70, Pt 12.22; MS/FAB molecular ion: 1663 m/z (M+ Na+).

### 5.4 Example 4

### Preparation of Liposomal Suspension of Complex 14

Lipids DMPC (1.57 g of) (Lipoid, GMBH) and sodium DMPG (0.68 g) (Lipoid, GMBH) were dissolved in a solution of tert-butanol (200 mL) containing Tween 20® (0.26 g) at 70° C. The solution was cooled to 35° C., and complex 14 (0.41 g) was added. The resultant mixture was stirred until dissolution was complete, and the solution was lyophilized. The resultant lyophilate was reconstituted with 0.9% Sodium Chloride Injection, USP (5 mg per milliliter) to provide a liposomal suspension of complex 14.

### 5.5 Example 5

### Use of Complex 14 to Identify Conversion Products in Liposomal Formulations of NDDP

Aroplatin™is a liposomal encapsulated diaminocyclohexane platinum compound containing cis-bis[neodecanoato-trans-(R,R-1,2-diaminocyclohexane)]platinum(II), (NDDP), (15). A liposomal suspension of complex 15 was prepared as described in Examples 3 and 5, and the reconstituted liposomal suspension sat for two days at room temperature, allowing the suspension to undergo conversion. The conversion product was dissolved in methanol and eluted on an HPLC column (reverse phase C18 column) equipped with ultraviolet light (UV) and evaporative light scattering (ELS) detectors. In a first method, the eluting solvent was 100% methanol; in a second method, the eluting solvent was methanol:water (80:20 v:v and increasing to 100% methanol). Elution times for the liposome suspension of complex 15 were 2.0 min and 9.6 min for the first and second methods, respectively.

For comparison, complex 14 (in its pure form as prepared in Example 3) was dissolved in methanol and analyzed by the HPLC method described above. Elution times for complex 14 were also 2.0 min and 9.6 min for the first and second methods, respectively.

While not intending to be bound by theory, Applicant believes that the results of the HPLC study suggest that complex 14 is a conversion product of the liposomal suspension of complex 15.

### 5.6 Example 6

### In Vitro Assays for Treatment or Prevention of Cancer

The following test can be used to assess the in vitro anticancer activity of liposomal suspensions of complex 14 in human and murine cell lines.

A liposomal suspension of complex 14 is added to human tumor cell lines (HT 29, B16, and PACA2) and murine tumor cell lines (L1210, CT26) established in 96-well plates. After 18 hours of incubation, the cells are pulsed with 3H thymidine and washed with phosphate-buffered saline (PBS). The amount of radioisotope incorporation is measured and used to calculate the inhibitory concentration 50 (IC50), which is the concentration that causes a 50% decrease in cell proliferation. The test protocol for the L1210 model is described in Han et al., Cancer Chemother. Pharmacol. 39: 17-24 (1996).

### 5.7 Example 7

### In Vivo Assay for Treatment or Prevention of Leukemia

The following test can be used to assess the in vivo antileukemia activity of liposomal suspensions of complex 14 against L1210 mouse leukemia.

Groups of 6-8 B6D2/F1 mice are challenged via the intraperitoneal (I.P.) route with 10⁶ L1210 cells. The animals are then treated via I.P. route with 5, 10 and 15 mg/kg of liposomal suspension of complex 14 or empty liposomes on days 1, 4 and 9 post-inoculation and the animals are scored twice daily to assess abdominal swelling—indicative of leukemia cell activity—and survival.

### 5.8 Example 8

### In Vitro Assay for Treatment or Prevention of Colorectal Cancer

The following test can be used to assess the in vivo activity of liposomal suspensions of complex 14 against colorectal cancer using HT29 human colorectal carcinoma cell xenografts.

Female BALB/c nude mice aged 6 weeks are injected subcutaneously with HT29 cells (10⁶). Treatment is started when tumors are measurable (mean tumor surface area at least 33 mm²). Four groups of mice are treated by injection into the tail vein: Group 1 is injected with diluent; Group 2 is injected with a liposomal formulation of complex 14 at 3, 5 and 10 mg/kg; Group 3 is injected with 5-Fluorouracil at 50 mg/kg; and Group 4 is injected with 5-Fluorouracil at 50 mg/kg followed by injection of liposomal formulation of platinum complex 14 at 3, 5 and 10 mg/kg. Tumor size measurements and body weights are taken daily. The test protocol is described in Raymond et al., Anti-Cancer Drugs 8: 876-885 (1997).

### 5.9 Example 9

### In Vivo Assay for Treatment Or Prevention of Liver Cancer

The following test can be used to assess the in vivo activity of liposomal suspensions of complex 14 against M5076 Reticulosarcoma liver metastases.

C57BL/6 mice are inoculated intravenously with M5076 cells (2×10⁴). Three groups of mice are treated by injection into the tail vein at day 4, 11 and 18: Group 1 is injected with diluent; Group 2 is injected with liposomal formulation of complex 14 at 3, 5 and 10 mg/kg; and Group 3 is injected with liposomal Aroplatin® at 20 mg/kg. The animals are sacrificed on day 30. The livers are dissected and placed in Bouin's solution (15 parts (by volume) of concentrated picric acid, 3 parts of 37-40% formalin, and 1 part glacial acetic acid). the number of tumor nodules on the liver surface are counted by 2 investigators. The test protocol is described in Perez-Solar et al., Cancer Res. 5: 6341-6347 (1992).

All references cited herein are incorporated herein by reference in their entirety and for all purposes to the same extent as if each individual publication or patent or patent application was specifically and individually indicated to be incorporated by reference in its entirety for all purposes.

Many modifications and variations of this invention can be made without departing from its spirit and scope, as will be apparent to those skilled in the art. The specific embodiments described herein are offered by way of example only, and the invention is to be limited only by the terms of the appended claims along with the full scope of equivalents to which such claims are entitled.

1. A purified lipid platinum complex having the formula (I): 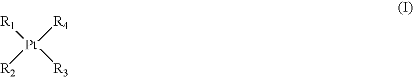
or a pharmaceutically acceptable salt thereof,
wherein
R₁ and R₂ are independently —N(R₆)₂, —NH₃⁺; or R₁ and R₂ are each —NH₂ and join through a C₂-C₆ alkylene or C₃-C₇ cycloalkylene group to form a bidentate diamine ligand, optionally substituted with one or more R₇;
R₃ is a lipid ligand, with the proviso that R₃ cannot be a phosphatidic acid;
R₄ is a lipid ligand, an inorganic ligand, —CN or —OC(O)R₅, with the proviso that R₄ cannot be a phosphatidic acid;
R₅ is C₁-C₂₄ alkyl;
each R₆ is independently —H, —C₁-C₆ alkyl, —C₃-C₇ cycloalkyl or -aryl; and
each R₇ is independently-C₁-C₆ alkyl, —C₃-C₇ cycloalkyl or -aryl.

2. The lipid platinum complex of claim 1 where R₃ is a phospholipid.

3. The lipid platinum complex of claim 1 where R₄ is a phospholipid.

4. The lipid platinum complex of claim 1 where R₃ and R₄ are each independently a phospholipid.

5. The complex of claim 2 where the phospholipid is a phosphatidyl choline, a phosphatidyl glycerol, a phosphatidyl ethanolamine or a sphingomyelin.

6. The complex of claim 2 where the phospholipid is dimyristoyl phosphatidyl choline, egg phosphatidyl choline, dilauryloyl phosphatidyl choline, dipalmitoyl phosphatidyl choline, distearoyl phosphatidyl choline, 1-myristoyl-2-palmitoyl phosphatidyl choline, 1-palmitoyl-2-myristoyl phosphatidyl choline, 1-palmitoyl-2-stearoyl phosphatidyl choline, 1-stearoyl-2-palmitoyl phosphatidyl choline or dioleoyl phosphatidyl choline.

7. The complex of claim 6 where the phospholipid is dimyristoyl phosphatidyl choline.

8. The complex of claim 2 where the phospholipid is dimyristoyl phosphatidyl glycerol, dilauryloyl phosphatidyl glycerol, dipalmitoyl phosphatidyl glycerol, distearoyl phosphatidyl glycerol, 1-myristoyl-2-palmitoyl phosphatidyl glycerol, 1-palmitoyl-2-myristoyl phosphatidyl glycerol, 1-palmitoyl-2-stearoyl phosphatidyl glycerol, 1-stearoyl-2-palmitoyl phosphatidyl glycerol and dioleoyl phosphatidyl glycerol.

9. The complex of claim 8 where the phospholipid is dimyristoyl phosphatidyl glycerol.

10-11. (canceled)

12. The lipid platinum complex of claim 1 where R₁ and R₂ join to form a bidentate diamine ligand.

13. The lipid platinum complex of claim 12 where the bidentate diamine ligand is trans-R,R-1,2-diaminocyclohexane, trans-S,S-1,2-diaminocyclohexane, cis-1,2-diaminocyclohexane or 1,2-ethylenediamine.

14. The lipid platinum complex of claim 13 where the bidendate diamine ligand is trans-R,R-1,2-diaminocyclohexane.

15. The lipid platinum complex of claim 1 where R₄ is an inorganic ligand, —CN or —OC(O)R₅; where R₅ is C₁-C₂₄ alkyl.

16. The lipid platinum complex of claim 1 where R₄ is Cl⁻, Br⁻, I⁻, F⁻, NO₃⁻, CN⁻, OH⁻, H₂O, HCO₃⁻ or HSO₄⁻.

17. The lipid platinum complex of claim 1 where R₄ is —OC(O)R₅, and R₅ has 5-11 carbon atoms.

18. The lipid platinum complex of claim 17 where R₅ has 9 carbon atoms.

19. The lipid platinum complex of claim 1 where R₄ is —OC(O)R₅ and R₅ is branched.

20. The lipid platinum complex of claim 1 where R₄ is —OC(O)R₅ and R₅ is linear.

21. The lipid platinum complex of claim 1 where R₄ is a neodecanoato group.

22. The lipid platinum complex of claim 1, wherein the lipid platinum complex is:
cis-bis[1,2-dimyristoyl-sn-glycero-3-phospho(rac-1-glycerol)][trans-(1R,2R)-1,2-diaminocyclohexane]platinum(II),
cis-[1,2-dimyristoyl-sn-glycero-3-phospho(rac-1-glycerol)] (neo-decanoato) [trans-(1R,2R)— 1,2-diaminocyclohexane]platinum(II),
cis-bis[1,2-dimyristoyl-sn-glycero-3-phospho(rac-1-glycerol)[trans-(1S,2S)-1,2-diaminocyclohexane]platinum (II),
cis-bis[1,2-dimyristoyl-sn-glycero-3-phospho(rac-1-glycerol)[cis-1,2-diaminocyclohexane]platinum (II),
cis-bis[1,2-dimyristoyl-sn-glycero-3-phospho(rac-1-glycerol)[trans-(rac)-diaminocyclohexane]platinum (II),
cis-[1,2-dimyristoyl-sn-glycero-3-phospho(rac-1-glycerol)(neo-decanoato)[trans-(1S,2S)-1,2-diaminocyclohexane]platinum (II),
cis-[1,2-dimyristoyl-sn-glycero-3-phospho(rac-1-glycerol)(neo-decanoato)[cis-1,2-diaminocyclohexane]platinum (II),
cis-[1,2-dimyristoyl-sn-glycero-3-phospho(rac-1-glycerol)(neo-decanoato)[trans-(rac)-1,2-diaminocyclohexane]platinum (II),
or a pharmaceutically acceptable salt thereof, said complex or salt being in purified form.

23-29. (canceled)

30. A liposomal lipid platinum complex comprising the lipid platinum complex of claim 1 entrapped within a liposome, said liposome comprising a liposomal lipid component, wherein the lipid platinum complex of claim 1 and the liposomal lipid component are present in a molar ratio between 1 to 2 and 1 to 30.

31. A liposomal lipid platinum complex comprising the lipid platinum complex of claim 22 entrapped within a liposome, said liposome comprising a liposomal lipid component, wherein the lipid platinum complex of claim 22 and the liposomal lipid component are present in a molar ratio between 1 to 2 and 1 to 7.

32. The liposomal lipid platinum complex of claim 30 where the lipid platinum complex of claim 1 and the liposomal lipid component are present in a molar ratio between 1 to 3 and 1 to 5.

33. The liposomal lipid platinum complex of claim 30 where the liposomal lipid component is dimyristoyl phosphatidyl glycerol, dimyristoyl phosphatidyl choline or a combination thereof.

34. The liposomal lipid platinum complex of claim 30 where the liposome is multilamellar.

35. The liposomal lipid platinum complex of claim 30 where the liposome is unilamellar.

36. The liposomal lipid platinum complex of claim 30 further comprising a surfactant.

37. The liposomal lipid platinum complex of claim 36 where the surfactant is present in an amount between 0.01 mole % and 4 mole % of the liposomal lipid component.

38. The liposomal lipid platinum complex of claim 36 where the surfactant is anionic, nonionic or cationic.

39-40. (canceled)

41. The liposomal lipid platinum complex of claim 38 where the surfactant is sorbitan polyoxyethylene carboxylate.

42. The liposomal lipid platinum complex of claim 38 where the surfactant is sorbitan polyoxyethylene monolaurate or sorbitan polyoxyethylene monooleate.

43. The liposomal lipid platinum complex of claim 36 having a median diameter of less than 1 μm.

44. The liposomal lipid platinum complex of claim 30 further comprising an additional anticancer agent other than the lipid platinum complex or a pharmaceutically acceptable salt of the lipid platinum complex.

45. The liposomal lipid platinum complex of claim 44 where the additional anticancer agent is gemcitabine, capecitabine or 5-fluorouracil.

46-47. (canceled)

48. A pharmaceutical composition comprising an amount of the lipid platinum complex of claim 1 or a pharmaceutically acceptable salt of the lipid platinum complex of claim 1, effective to treat cancer, and a pharmaceutically acceptable carrier or vehicle.

49. The pharmaceutical composition of claim 48 further comprising an amount of an additional anticancer agent other than the lipid platinum complex or a pharmaceutically acceptable salt of the lipid platinum complex, effective to treat cancer.

50. The pharmaceutical composition of claim 49 where the additional anticancer agent is gemcitabine, capecitabine or 5-fluorouracil.

51. A pharmaceutical composition comprising an amount of the lipid platinum complex of claim 22 or a pharmaceutically acceptable salt of the lipid platinum complex of claim 22, effective to treat cancer, and a pharmaceutically acceptable carrier or vehicle.

52. The pharmaceutical composition of claim 51 further comprising an amount of an additional anticancer agent other than the lipid platinum complex or a pharmaceutically acceptable salt of the lipid platinum complex, effective to treat cancer.

53. The pharmaceutical composition of claim 52 where the additional anticancer agent is gemcitabine, capecitabine or 5-fluorouracil.

54-59. (canceled)

60. A method for treating cancer, the method comprising administering to a subject in need thereof an amount of the lipid platinum complex of claim 1 or a pharmaceutically acceptable salt of the lipid platinum complex of claim 1, effective to treat cancer.

61. A method for treating cancer, the method comprising administering to a subject in need thereof an amount of the lipid platinum complex of claim 22 or a pharmaceutically acceptable salt thereof, effective to treat cancer.

62. A method for treating cancer, the method comprising administering to a subject in need thereof an amount of the liposomal lipid platinum complex of claim 30 effective to treat cancer.

63. A method for treating cancer, the method comprising administering to a subject in need thereof an amount of the liposomal lipid platinum complex of claim 31 effective to treat cancer.

64. A method for treating cancer, the method comprising administering to a subject in need thereof the pharmaceutical composition of claim 48.

65. A method for treating cancer, the method comprising administering to a subject in need thereof the pharmaceutical composition of claim 51.

66-67. (canceled)

68. The method of claim 60 further comprising administering to said subject an additional anticancer agent which is not the lipid platinum complex or the pharmaceutically acceptable salt of the lipid platinum complex.

69. The method of claim 62 further comprising administering to said subject an additional anticancer agent which is not the liposomal lipid platinum complex.

70. The method of claim 68 wherein the additional anticancer agent is gemcitabine, capecitabine or 5-fluorouracil.

71. The method of claim 69 wherein the additional anticancer agent is gemcitabine, capecitabine or 5-fluorouracil.

72. The method of claim 60 wherein the cancer is pancreatic cancer, colorectal cancer or mesothelioma.

73. The method of claim 60 wherein the subject is a human.

74. A kit comprising a container which contains a unit dosage form of the lipid platinum complex of claim 1 or a pharmaceutically acceptable salt thereof.

75. A kit comprising a container which contains a unit dosage form of the liposomal lipid platinum complex of claim 30.

76. The kit of claim 75 where the liposomal lipid platinum complex is in lyophilized form.

77. The kit of claim 76 further comprising a second container, the second container containing a solution useful for reconstitution of the lyophilized liposomal lipid platinum complex.

78. The kit of claim 77 where the solution is an aqueous solution.

79. The kit of claim 78 where the aqueous solution comprises sodium chloride.

80. The kit of claim 79 where the aqueous solution is a saline solution.

81. The kit of claim 80 where the saline solution is phosphate buffered saline.

82. The kit of claim 74 further comprising a second container, the second container containing an additional anticancer agent other than the lipid platinum complex or a pharmaceutically acceptable salt of the lipid platinum complex.

83. The kit of claim 82 where the additional anticancer agent is gemcitabine, capecitabine or 5-fluorouracil.

84. A kit comprising a first container which contains a unit dosage form of the liposomal lipid platinum complex of claim 30, and a second container, the second container containing an additional anticancer agent other than the liposomal lipid platinum complex.

85. The kit of claim 84 where the additional anticancer agent is gemcitabine, capecitabine or 5-fluorouracil.

86. The kit of claim 74 further comprising a second container, the second container containing an antiemetic agent or a hematopoietic colony stimulating factor.

87. The kit of claim 74 further comprising means for administering the lipid platinum complex or a pharmaceutically acceptable salt thereof to a subject.

88. A kit comprising a container which contains a unit dosage form of the liposomal lipid platinum complex of claim 30 and means for administering the liposomal lipid platinum complex to a subject.

89. A method for making a platinum complex of formula (I), 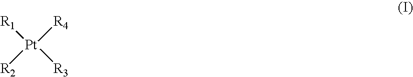
comprising allowing a complex of formula(II), 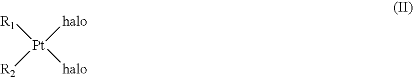
to react with at least about 2 molar equivalents of the silver salt of 1,2-dimyristoyl-sn-glycero-3-phospho(rac-1-glycerol)
wherein
R₁ and R₂ form trans-R,R-1,2-diaminocyclohexane;
R₃ is 1,2-dimyristoyl-sn-glycero-3-phospho(rac-1-glycerol); and
R₄ is 1,2-dimyristoyl-sn-glycero-3-phospho(rac-1-glycerol).

90-97. (canceled)

98. The method of claim 64 further comprising administering to said subject an additional anticancer agent which is not the lipid platinum complex or the pharmaceutically acceptable salt of the lipid platinum complex.

99. The method of claim 66 further comprising administering to said subject an additional anticancer agent which is not the liposomal lipid platinum complex.

100. The lipid platinum complex of claim 1, wherein
R₁ and R₂ are independently —N(R₆)₂, —NH₃⁺; or R₁ and R₂ are each —NH₂ and join through a C₂-C₆ alkylene or C₃-C₇ cycloalkylene group to form a bidentate diamine ligand;
R₃ is a lipid ligand, with the proviso that R₃ cannot be a phosphatidic acid;
R₄ is a lipid ligand, an inorganic ligand, —CN or —OC(O)R₅, with the proviso that R₄ cannot be a phosphatidic acid;
R₅ is C₁-C₂₄ alkyl;
each R₆ is independently —H, —C₁-C₆ alkyl, —C₃-C₇ cycloalkyl or -aryl.

101. The liposomal lipid platinum complex of claim 30, wherein the lipid platinum complex and the liposomal lipid component are present in a molar ratio between 1 to 2 and 1 to 7.

102. The liposomal lipid platinum complex of claim 37 where the surfactant is present in an amount between 0.5 mole % and 4 mole % of the liposomal lipid component.
